(19) United States
(12) Patent Application Publication
(10) Pub. No.: US 2011/0082484 A1
(43) Pub. Date: Apr. 7, 2011

(54) SENSOR INSERTER ASSEMBLY HAVING ROTATABLE TRIGGER

(76) Inventors: Heber Saravia, San Francisco, CA (US); Daniel H. Lee, Burlingame, CA (US)

(21) Appl. No.: 12/900,363

(22) Filed: Oct. 7, 2010

Related U.S. Application Data
(60) Provisional application No. 61/249,535, filed on Oct. 7, 2009.

(51) Int. Cl. A61B 17/32

(52) U.S. Cl. 606/167

(57) ABSTRACT

An inserter subassembly that is engaged by turning a rotatable trigger to implant the analyte sensor.

### RELATED APPLICATIONS

The present application claims the benefit of U.S. Provisional Application No. 61/249,535, filed Oct. 7, 2009, the disclosure of which is incorporated herein by reference for all purposes.

### FIELD OF THE INVENTION

The present invention relates generally to an inserter device, for example, to insert an analyte sensor or an infusion set. More specifically, the present invention relates to an inserter device comprising a rotatable trigger.

### BACKGROUND OF THE INVENTION

The detection and/or monitoring of glucose levels or other analytes, such as lactate, oxygen, A1C, or the like, in certain individuals is vitally important to their health. For example, the monitoring of glucose is particularly important to individuals with diabetes. Diabetics generally monitor glucose levels to determine if their glucose levels are being maintained within a clinically safe range, and may also use this information to determine if and/or when insulin is needed to reduce glucose levels in their bodies or when additional glucose is needed to raise the level of glucose in their bodies.

Growing clinical data demonstrates a strong correlation between the frequency of glucose monitoring and glycemic control. Despite such correlation, many individuals diagnosed with a diabetic condition do not monitor their glucose levels as frequently as they should due to a combination of factors including convenience, testing discretion, pain associated with glucose testing, and cost.

Devices have been developed for the automatic monitoring of analyte(s), such as glucose, in bodily fluid such as in the blood stream or in interstitial fluid (“ISF”), or other biological fluid. Some of these analyte measuring devices are configured so that at least a portion of the devices are positioned below a skin surface of a user, e.g., in a blood vessel or in the subcutaneous tissue of a user, so that the monitoring is accomplished in vivo.

With the continued development of analyte monitoring devices and systems, there is a need for such analyte monitoring devices, systems, and methods, as well as devices for inserting and/or positioning such analyte monitoring systems that are cost effective, convenient, and with reduced pain, provide discreet monitoring to encourage frequent analyte monitoring to improve glycemic control.

### SUMMARY

In one aspect of the invention, a sensor inserter assembly is provided. The inserter assembly includes a housing and a shuttle movably connected to the housing. The shuttle can move in an insertion direction. In this regard, a driver can be included to urge the shuttle in the insertion direction. In some embodiments, a second spring can be included for urging the shuttle in a retraction direction.

In some embodiments, the sensor inserter assembly is pre-loaded with a sensor. The sensor can be received in an introducer sharp, which can be attached to the shuttle. The sensor inserter assembly further comprises a rotatable trigger that can be axially received on the tubular housing. The tubular housing can include a groove circumferentially disposed about the tubular body to engage with a flange disposed on the trigger.

In some embodiments, the housing includes one or more centrally located channels extending through the tubular body. A centrally located aperture can be formed in the tubular body to receive the shuttle and introducer sharp. The first spring can be disposed proximal to the shuttle and introducer sharp in the centrally located channel of tubular housing. In this manner, the rotatable trigger can be configured to release the shuttle and allow the first spring means to urge the shuttle and the introducer sharp in the insertion direction. The tubular housing can further include a second spring to facilitate retraction of the shuttle and introducer sharp in the retraction direction.

In some embodiments, the rotatable trigger is configured to release the shuttle from a mounting unit. For example, the rotatable trigger can be configured to rotate 180 degrees to release the shuttle. The inserter can be configured to insert the sensor at an angle. For example, in some embodiments, the sensor is inserted at a 20 degree angle relative to the user's skin.

In some embodiments, the introducer includes one or more cantilever arms to retain the sensor. Further, the sensor can be retained in the introducer of the sensor inserter assembly by various structures, including a dimple disposed on the sensor body and configured to form an interference fit with the introducer.

### BRIEF DESCRIPTION OF THE DRAWINGS

A detailed description of various aspects, features, and embodiments of the subject matter described herein is provided with reference to the accompanying drawings, which are briefly described below. The drawings are illustrative and are not necessarily drawn to scale, with some components and features being exaggerated for clarity. The drawings illustrate various aspects and features of the present subject matter and may illustrate one or more embodiment(s) or example(s) of the present subject matter in whole or in part.

FIG. 1 illustrates a schematic view of a data monitoring and management system for practicing one or more embodiments of the present invention.

FIG. 2 illustrates an inserter and mount assembly for practicing one or more embodiments of the present invention.

FIG. 3 illustrates an exploded view of the inserter of FIG. 2.

FIGS. 4A-4C illustrate the steps used to assemble the shuttle and introducer sharp.

FIGS. 5A-5B illustrate the steps used to load the retraction spring into the inserter.

FIGS. 6A-6C illustrate the steps used to load the shuttle into the inserter.

FIG. 7A-7B illustrate the steps used to insert the driver spring into the inserter.

FIGS. 8A-8D illustrate the steps used to attach the rotatable trigger to the inserter.

FIGS. 9A-9E illustrate the steps used to load the sensor into the inserter.

FIG. 9F illustrates the sensor loaded into the inserter.

FIG. 9G illustrates the components of the sensor.

FIGS. 10A-10C illustrate the steps used to prepare the inserter for firing.

FIGS. 11A-11B illustrate the steps used to attach the seal to the mount using an adhesive or welding method.

FIGS. 12A-12D illustrate the steps used to insert the battery and contact into the mount.

FIGS. 13A-13B illustrate the steps used to attach the adhesive assembly to the mount.

FIGS. 14A-14C illustrate the steps used to attach the thermocouple to the base of the on body electronics unit.

FIGS. 15A-15B illustrate the steps used to attach the printed circuit board to the lid of the on body electronics unit.

FIGS. 16A-16B illustrate the steps to attach the thermocouple from the base to the lid of the on body electronics unit.

FIGS. 17A-17D illustrate the steps used to attach the base to the lid of the on body electronics unit.

FIGS. 17E-17G illustrate the steps used to attach the on body electronics unit to the mount.

FIGS. 18A-18H illustrate the steps used to attach the inserter to the mount.

FIG. 19 illustrates the on body electronics unit attached to the mount with the sensor deployed.

FIG. 20 the mount and on body electronics unit attached to a user.

### DETAILED DESCRIPTION OF EXEMPLARY EMBODIMENTS

Before the present disclosure is described in detail, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges is also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described. All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise.

The publications discussed herein are provided solely for their disclosure prior to the filing date of the present application. Nothing herein is to be construed as an admission that the present disclosure is not entitled to antedate such publication by virtue of prior disclosure.

Further, the dates of publication provided may be different from the actual publication dates which may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

The figures shown herein are not necessarily drawn to scale, with some components and features being exaggerated for clarity.

Generally, embodiments of the present disclosure relate to in vivo methods and devices for detecting at least one analyte such as glucose in body fluid. Accordingly, embodiments include in vivo analyte sensors configured so that at least a portion of the sensor is positioned in the body of a user (e.g., within the ISF), to obtain information about at least one analyte of the body, e.g., transcutaneously positioned in user's body. In certain embodiments, an in vivo analyte sensor is coupled to an electronics unit that is maintained on the body of the user such as on a skin surface, where such coupling provides on body, in vivo analyte sensor electronics assemblies.

In certain embodiments, analyte information is communicated from a first device such as an on body electronics unit to a second device which may include user interface features, including a display, and/or the like. Information may be communicated from the first device to the second device automatically and/or continuously when the analyte information is available, or may not be communicated automatically and/or continuously, but rather stored or logged in a memory of the first device. Accordingly, in many embodiments of the system, analyte information derived by the sensor/on body electronics (for example, on body electronics assembly) is made available in a user-usable or viewable form only when queried by the user such that the timing of data communication is selected by the user.

In this manner, analyte information is only provided or evident to a user (provided at a user interface device) when desired by the user even though an in vivo analyte sensor automatically and/or continuously monitors the analyte level in vivo, i.e., the sensor automatically monitors analyte such as glucose on a pre-defined time interval over its usage life. For example, an analyte sensor may be positioned in vivo and coupled to on body electronics for a given sensing period, e.g., about 14 days. In certain embodiments, the sensor-derived analyte information is automatically communicated from the sensor electronics assembly to a remote monitor device or display device for output to a user throughout the 14 day period according to a schedule programmed at the on body electronics (e.g., about every 1 minute or about every 5 minutes or about every 10 minutes, or the like). In certain embodiments, sensor-derived analyte information is only communicated from the sensor electronics assembly to a remote monitor device or display device at user-determined times, e.g., whenever a user decides to check analyte information. At such times, a communications system is activated and sensor-derived information is then sent from the on body electronics to the remote device or display device.

In still other embodiments, the information may be communicated from the first device to the second device automatically and/or continuously when the analyte information is available, and the second device stores or logs the received information without presenting or outputting the information to the user. In such embodiments, the information is received by the second device from the first device when the information becomes available (e.g., when the sensor detects the analyte level according to a time schedule). However, the received information is initially stored in the second device and only output to a user interface or an output component of the second device (e.g., display) upon detection of a request for the information on the second device.

Accordingly, in certain embodiments once a sensor electronics assembly is placed on the body so that at least a portion of the in vivo sensor is in contact with bodily fluid such as ISF and the sensor is electrically coupled to the electronics unit, sensor derived analyte information may be communicated from the on body electronics to a display device on-demand by powering on the display device (or it may be continually powered), and executing a software algorithm stored in and accessed from a memory of the display device, to generate one or more request commands, control signal or data packet to send to the on body electronics. The software algorithm executed under, for example, the control of the microprocessor or application specific integrated circuit (ASIC) of the display device may include routines to detect the position of the on body electronics relative to the display device to initiate the transmission of the generated request command, control signal and/or data packet.

Display devices may also include programming stored in memory for execution by one or more microprocessors and/or ASICs to generate and transmit the one or more request command, control signal or data packet to send to the on body electronics in response to a user activation of an input mechanism on the display device such as depressing a button on the display device, triggering a soft button associated with the data communication function, and so on. The input mechanism may be alternatively or additionally provided on or in the on body electronics which may be configured for user activation. In certain embodiments, voice commands or audible signals may be used to prompt or instruct the microprocessor or ASIC to execute the software routine(s) stored in the memory to generate and transmit the one or more request command, control signal or data packet to the on body device. In the embodiments that are voice activated or responsive to voice commands or audible signals, on body electronics and/or display device includes a microphone, a speaker, and processing routines stored in the respective memories of the on body electronics and/or the display device to process the voice commands and/or audible signals. In certain embodiments, positioning the on body device and the display device within a predetermined distance (e.g., close proximity) relative to each other initiates one or more software routines stored in the memory of the display device to generate and transmit a request command, control signal or data packet.

Different types and/or forms and/or amounts of information may be sent for each on demand reading, including but not limited to one or more of current analyte level information (i.e., real time or the most recently obtained analyte level information temporally corresponding to the time the reading is initiated), rate of change of an analyte over a predetermined time period, rate of the rate of change of an analyte (acceleration in the rate of change), historical analyte information corresponding to analyte information obtained prior to a given reading and stored in memory of the assembly. Some or all of real time, historical, rate of change, rate of rate of change (such as acceleration or deceleration) information may be sent to a display device for a given reading. In certain embodiments, the type and/or form and/or amount of information sent to a display device may be preprogrammed and/or unchangeable (e.g., preset at manufacturing), or may not be preprogrammed and/or unchangeable so that it may be selectable and/or changeable in the field one or more times (e.g., by activating a switch of the system, etc). Accordingly, in certain embodiments, for each on demand reading, a display device will output a current (real time) sensor-derived analyte value (e.g., in numerical format), a current rate of analyte change (e.g., in the form of an analyte rate indicator such as a arrow pointing in a direction to indicate the current rate), and analyte trend history data based on sensor readings acquired by and stored in memory of on body electronics (e.g., in the form of a graphical trace). Additionally, the on skin or sensor temperature reading or measurement associated with each on demand reading may be communicated from the on body electronics to the display device. The temperature reading or measurement, however, may not be output or displayed on the display device, but rather, used in conjunction with a software routine executed by the display device to correct or compensate the analyte measurement output to the user on the display device.

As described, embodiments include in vivo analyte sensors and on body electronics that together provide body wearable sensor electronics assemblies. In certain embodiments, in vivo analyte sensors are fully integrated with on body electronics (fixedly connected during manufacture), while in other embodiments they are separate but connectable post manufacture (e.g., before, during or after sensor insertion into a body). On body electronics may include an in vivo glucose sensor, electronics, battery, and antenna encased (except for the sensor portion that is for in vivo positioning) in a waterproof housing that includes or is attachable to an adhesive pad. In certain embodiments, the housing withstands immersion in about one meter of water for up to at least 30 minutes. In certain embodiments, the housing withstands continuous underwater contact, e.g., for longer than about 30 minutes, and continues to function properly according to its intended use, e.g., without water damage to the housing electronics where the housing is suitable for water submersion.

Embodiments include sensor insertion devices, which also may be referred to herein as sensor delivery units, or the like. Insertion devices may retain on body electronics assemblies completely in an interior compartment, i.e., an insertion device may be “pre-loaded” with on body electronics assemblies during the manufacturing process (e.g., on body electronics may be packaged in a sterile interior compartment of an insertion device). In such embodiments, insertion devices may form sensor assembly packages (including sterile packages) for pre-use or new on body electronics assemblies, and insertion devices configured to apply on body electronics assemblies to recipient bodies.

Embodiments include portable handheld display devices, as separate devices and spaced apart from an on body electronics assembly, that collect information from the assemblies and provide sensor derived analyte readings to users. Such devices may also be referred to as meters, readers, monitors, receivers, human interface devices, companions, or the like. Certain embodiments may include an integrated in vitro analyte meter. In certain embodiments, display devices include one or more wired or wireless communications ports such as USB, serial, parallel, or the like, configured to establish communication between a display device and another unit (e.g., on body electronics, power unit to recharge a battery, a PC, etc). For example, a display device communication port may enable charging a display device battery with a respective charging cable and/or data exchange between a display device and its compatible informatics software.

Compatible informatics software in certain embodiments include, for example, but not limited to stand alone or network connection enabled data management software program, resident or running on a display device, personal computer, a server terminal, for example, to perform data analysis, charting, data storage, data archiving and data communication as well as data synchronization. Informatics software in certain embodiments may also include software for executing field upgradable functions to upgrade firmware of a display device and/or on body electronics unit to upgrade the resident software on the display device and/or the on body electronics unit, e.g., with versions of firmware that include additional features and/or include software bugs or errors fixed, etc.

Embodiments may include a haptic feedback feature such as a vibration motor or the like, configured so that corresponding notifications (e.g., a successful on-demand reading received at a display device), may be delivered in the form of haptic feedback.

Embodiments include programming embedded on a computer readable medium, i.e., computer-based application software (may also be referred to herein as informatics software or programming or the like) that processes analyte information obtained from the system and/or user self-reported data. Application software may be installed on a host computer such as a mobile telephone, PC, an Internet-enabled human interface device such as an Internet-enabled phone, personal digital assistant, or the like, by a display device or an on body electronics unit. Informatics programming may transform data acquired and stored on a display device or on body unit for use by a user.

Embodiments of the subject disclosure are described primarily with respect to glucose monitoring devices and systems, and methods of glucose monitoring, for convenience only and such description is in no way intended to limit the scope of the disclosure. It is to be understood that the analyte monitoring system may be configured to monitor a variety of analytes at the same time or at different times.

For example, analytes that may be monitored include, but are not limited to, acetyl choline, amylase, bilirubin, cholesterol, chorionic gonadotropin, creatine kinase (e.g., CK-MB), creatine, DNA, fructosamine, glucose, glutamine, growth hormones, hormones, ketones, lactate, oxygen, peroxide, prostate-specific antigen, prothrombin, RNA, thyroid stimulating hormone, and troponin. The concentration of drugs, such as, for example, antibiotics (e.g., gentamicin, vancomycin, and the like), digitoxin, digoxin, drugs of abuse, theophylline, and warfarin, may also be monitored. In those embodiments that monitor more than one analyte, the analytes may be monitored at the same or different times, with a single sensor or with a plurality of sensors which may use the same on body electronics (e.g., simultaneously) or with different on body electronics.

As described in detail below, embodiments include devices, systems, kits and/or methods to monitor one or more physiological parameters such as, for example, but not limited to, analyte levels, temperature levels, heart rate, user activity level, over a predetermined monitoring time period. Also provided are methods of manufacturing. Predetermined monitoring time periods may be less than about 1 hour, or may include about 1 hour or more, e.g., about a few hours or more, e.g., about a few days of more, e.g., about 3 or more days, e.g., about 5 days or more, e.g., about 7 days or more, e.g., about 10 days or more, e.g., about 14 days or more, e.g., about several weeks, e.g., about 1 month or more. In certain embodiments, after the expiration of the predetermined monitoring time period, one or more features of the system may be automatically deactivated or disabled at the on body electronics assembly and/or display device.

For example, a predetermined monitoring time period may begin with positioning the sensor in vivo and in contact with a body fluid such as ISF, and/or with the initiation (or powering on to full operational mode) of the on body electronics. Initialization of on body electronics may be implemented with a command generated and transmitted by a display device in response to the activation of a switch and/or by placing the display device within a predetermined distance (e.g., close proximity) to the on body electronics, or by user manual activation of a switch on the on body electronics unit, e.g., depressing a button, or such activation may be caused by the insertion device, e.g., as described in U.S. patent application Ser. No. 12/698,129 filed on Feb. 1, 2010 and U.S. Provisional Application Nos. 61/238,646, 61/246,825, 61/247,516, 61/249,535, 61/317,243, 61/345,562, and 61/361,374, the disclosures of each of which are incorporated herein by reference for all purposes.

When initialized in response to a received command from a display device, the on body electronics retrieves and executes from its memory software routine to fully power on the components of the on body electronics, effectively placing the on body electronics in full operational mode in response to receiving the activation command from the display device. For example, prior to the receipt of the command from the display device, a portion of the components in the on body electronics may be powered by its internal power supply such as a battery while another portion of the components in the on body electronics may be in powered down or low power including no power, inactive mode, or all components may be in an inactive mode, powered down mode. Upon receipt of the command, the remaining portion (or all) of the components of the on body electronics is switched to active, fully operational mode.

Embodiments of on body electronics may include one or more circuit boards with electronics including control logic implemented in ASIC, microprocessors, memory, and the like, and transcutaneously positionable analyte sensors forming a single assembly. On body electronics may be configured to provide one or more signals or data packets associated with a monitored analyte level upon detection of a display device of the analyte monitoring system within a predetermined proximity for a period of time (for example, about 2 minutes, e.g., 1 minute or less, e.g., about 30 seconds or less, e.g., about 10 seconds or less, e.g., about 5 seconds or less, e.g., about 2 seconds or less) and/or until a confirmation, such as an audible and/or visual and/or tactile (e.g., vibratory) notification, is output on the display device indicating successful acquisition of the analyte related signal from the on body electronics. A distinguishing notification may also be output for unsuccessful acquisition in certain embodiments.

In certain embodiments, the monitored analyte level may be correlated and/or converted to glucose levels in blood or other fluids such as ISF. Such conversion may be accomplished with the on body electronics, but in many embodiments will be accomplished with display device electronics. In certain embodiments, glucose level is derived from the monitored analyte level in the ISF.

Analyte sensors may be insertable into a vein, artery, or other portion of the body containing analyte. In certain embodiments, analyte sensors may be positioned in contact with ISF to detect the level of analyte, where the detected analyte level may be used to infer the user's glucose level in blood or interstitial tissue.

Embodiments include transcutaneous sensors and also wholly implantable sensors and wholly implantable assemblies in which a single assembly including the analyte sensor and electronics are provided in a sealed housing (e.g., hermetically sealed biocompatible housing) for implantation in a user's body for monitoring one or more physiological parameters.

### Embodiments of In Vivo Analyte Monitoring Systems

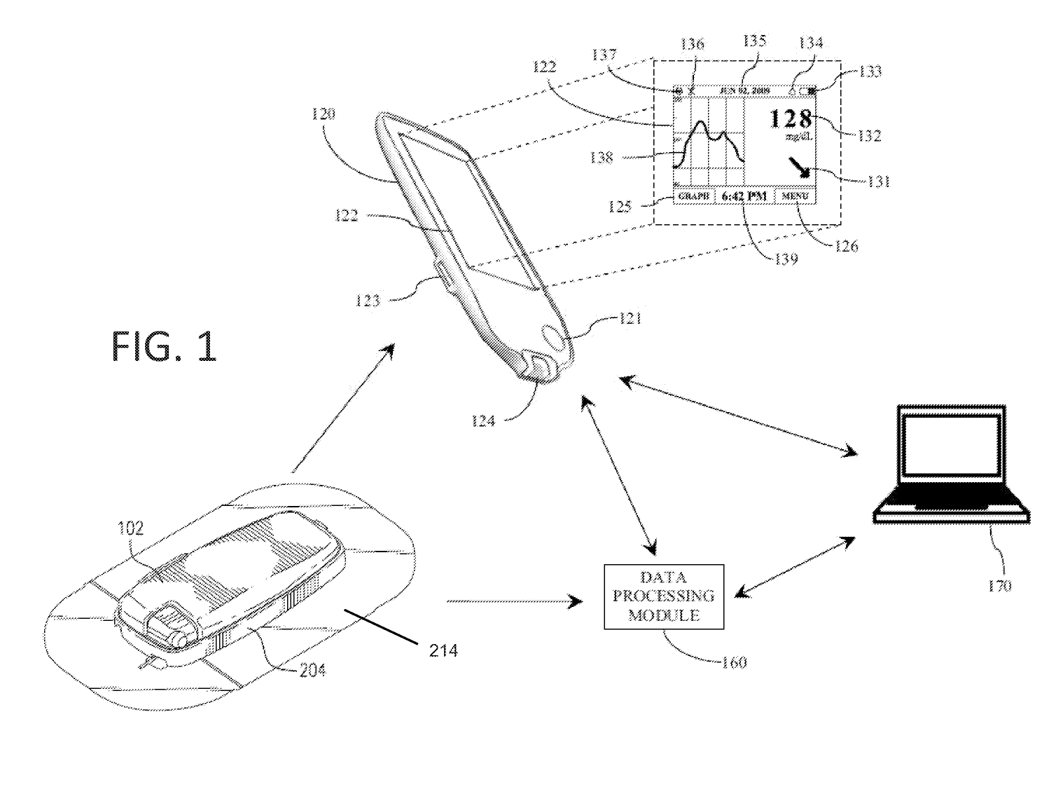

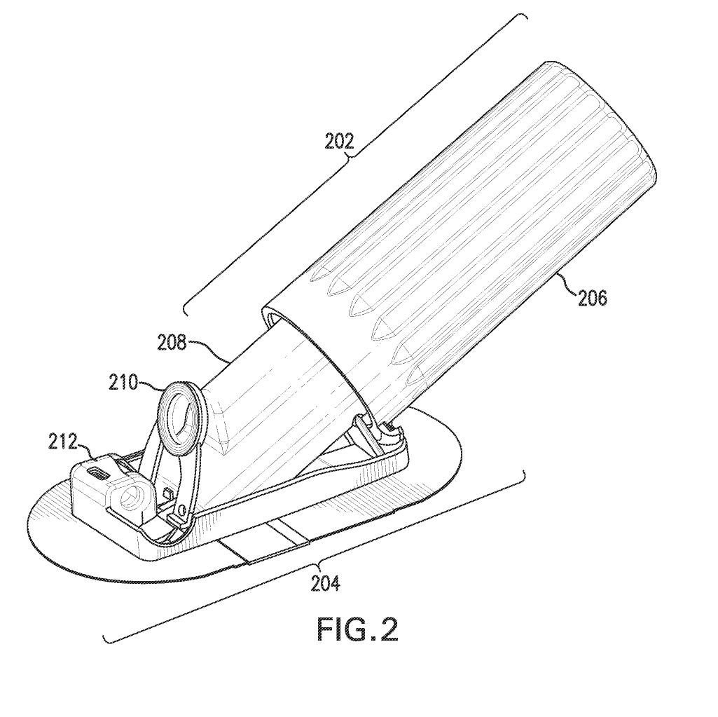

FIG. 1 shows an exemplary in vivo-based analyte monitoring system 100 in accordance with embodiments of the present disclosure. As shown, in certain embodiments, analyte monitoring system 100 includes on body electronics 102 electrically coupled to in vivo analyte sensor 302 (not shown in FIG. 1) and attached to adhesive layer 214 for attachment on a skin surface on the body of a user. On body electronics 102 includes on body housing, that defines an interior compartment. An inserter assembly 202 is illustrated in FIG. 2 that, when operated, transcutaneously positions a portion of analyte sensor 302 through a skin surface and in fluid contact with ISF. Devices, systems and methods that maybe used with embodiments herein are described, e.g., in U.S. patent application Ser. Nos. 12/807,278, 12/698,129 and U.S. Provisional Application Nos. 61/238,646, 61/246,825, 61/247,516, 61/249,535, 61/317,243, 61/345,562, and 61/361,374, the disclosures of each of which are incorporated herein by reference for all purposes.

Referring back to the FIG. 1, analyte monitoring system 100 includes display device 120 which includes a display 122 to output information to the user, an input component 121 such as a button, actuator, a touch sensitive switch, a capacitive switch, pressure sensitive switch, jog wheel or the like, to input data or command to display device 120 or otherwise control the operation of display device 120. It is noted that some embodiments may include display-less devices or devices without any user interface components. These devices may be functionalized to store data as a data logger and/or provide a conduit to transfer data from on body electronics and/or a display-less device to another device and/or location. Embodiments will be described herein as display devices for exemplary purposes which are in no way intended to limit the embodiments of the present disclosure. It will be apparent that display-less devices may also be used in certain embodiments.

In certain embodiments, on body electronics 102 may be configured to store some or all of the monitored analyte related data received from analyte sensor 302 in a memory during the monitoring time period, and maintain it in memory until the usage period ends. In such embodiments, stored data is retrieved from on body electronics 102 at the conclusion of the monitoring time period, for example, after removing analyte sensor 302 from the user by detaching on body electronics 102 from the skin surface where it was positioned during the monitoring time period. In such data logging configurations, real time monitored analyte level is not communicated to display device 120 during the monitoring period or otherwise transmitted from on body electronics 102, but rather, retrieved from on body electronics 102 after the monitoring time period.

In certain embodiments, input component 121 of display device 120 may include a microphone and display device 120 may include software configured to analyze audio input received from the microphone, such that functions and operation of the display device 120 may be controlled by voice commands. In certain embodiments, an output component of display device 120 includes a speaker for outputting information as audible signals. Similar voice responsive components such as a speaker, microphone and software routines to generate, process and store voice driven signals may be provided to on body electronics 102.

In certain embodiments, display 122 and input component 121 may be integrated into a single component, for example a display that can detect the presence and location of a physical contact touch upon the display such as a touch screen user interface. In such embodiments, the user may control the operation of display device 120 by utilizing a set of pre-programmed motion commands, including, but not limited to, single or double tapping the display, dragging a finger or instrument across the display, motioning multiple fingers or instruments toward one another, motioning multiple fingers or instruments away from one another, etc. In certain embodiments, a display includes a touch screen having areas of pixels with single or dual function capacitive elements that serve as LCD elements and touch sensors.

Display device 120 also includes data communication port 123 for wired data communication with external devices such as remote terminal (personal computer) 170, for example. Example embodiments of the data communication port 123 include USB port, mini USB port, RS-232 port, Ethernet port, Firewire port, or other similar data communication ports configured to connect to the compatible data cables. Display device 120 may also include an integrated in vitro glucose meter, including in vitro test strip port 124 to receive an in vitro glucose test strip for performing in vitro blood glucose measurements.

Referring still to FIG. 1, display 122 in certain embodiments is configured to display a variety of information—some or all of which may be displayed at the same or different time on display 122. In certain embodiments the displayed information is user-selectable so that a user can customize the information shown on a given display screen. Display 122 may include but is not limited to graphical display 138, for example, providing a graphical output of glucose values over a monitored time period (which may show important markers such as meals, exercise, sleep, heart rate, blood pressure, etc, numerical display 132, for example, providing monitored glucose values (acquired or received in response to the request for the information), and trend or directional arrow display 131 that indicates a rate of analyte change and/or a rate of the rate of analyte change, e.g., by moving locations on display 122.

As further shown in FIG. 1, display 222 may also include date display 135 providing for example, date information for the user, time of day information display 139 providing time of day information to the user, battery level indicator display 133 which graphically shows the condition of the battery (rechargeable or disposable) of the display device 120, sensor calibration status icon display 134 for example, in monitoring systems that require periodic, routine or a predetermined number of user calibration events, notifying the user that the analyte sensor calibration is necessary, audio/vibratory settings icon display 136 for displaying the status of the audio/vibratory output or alarm state, and wireless connectivity status icon display 137 that provides indication of wireless communication connection with other devices such as on body electronics, data processing module 160, and/or remote terminal 170. As additionally shown in FIG. 1, display 122 may further include simulated touch screen button 125, 126 for accessing menus, changing display graph output configurations or otherwise for controlling the operation of display device 120.

Referring to FIG. 1, in certain embodiments, display 122 of display device 120 may be additionally, or instead of visual display, configured to output alarms notifications such as alarm and/or alert notifications, glucose values etc, which may be audible, tactile, or any combination thereof. In one aspect, the display device 120 may include other output components such as a speaker, vibratory output component and the like to provide audible and/or vibratory output indication to the user in addition to the visual output indication provided on display 122. Further details and other display embodiments can be found in, e.g., U.S. patent application Ser. No. 12/871,901, U.S. provisional application Nos. 61/238,672, 61/247,541, 61/297,625, the disclosures of each of which are incorporated herein by reference for all purposes.

After the positioning of on body electronics 102 on the skin surface and analyte sensor 302 in vivo to establish fluid contact with interstitial fluid (or other appropriate body fluid), on body electronics 102 in certain embodiments is configured to wirelessly communicate analyte related data (such as, for example, data corresponding to monitored analyte level and/or monitored temperature data, and/or stored historical analyte related data) when on body electronics 102 receives a command or request signal from display device 120. In certain embodiments, on body electronics 102 may be configured to at least periodically broadcast real time data associated with monitored analyte level which is received by display device 120 when display device 120 is within communication range of the data broadcast from on body electronics 102, i.e., it does not need a command or request from a display device to send information.

For example, display device 120 may be configured to transmit one or more commands to on body electronics 102 to initiate data transfer, and in response, on body electronics 102 may be configured to wirelessly transmit stored analyte related data collected during the monitoring time period to display device 120. Display device 120 may in turn be connected to a remote terminal 170 such as a personal computer and functions as a data conduit to transfer the stored analyte level information from the on body electronics 102 to remote terminal 170. In certain embodiments, the received data from the on body electronics 102 may be stored (permanently or temporarily) in one or more memory of the display device 120. In certain other embodiments, display device 120 is configured as a data conduit to pass the data received from on body electronics 102 to remote terminal 170 that is connected to display device 120.

Referring still to FIG. 1, also shown in analyte monitoring system 100 are data processing module 160 and remote terminal 170. Remote terminal 170 may include a personal computer, a server terminal a laptop computer or other suitable data processing devices including software for data management and analysis and communication with the components in the analyte monitoring system 100. For example, remote terminal 170 may be connected to a local area network (LAN), a wide area network (WAN), or other data network for uni-directional or bi-directional data communication between remote terminal 170 and display device 120 and/or data processing module 160.

Remote terminal 170 in certain embodiments may include one or more computer terminals located at a physician's office or a hospital. For example, remote terminal 170 may be located at a location other than the location of display device 120. Remote terminal 170 and display device 120 could be in different rooms or different buildings. Remote terminal 170 and display device 120 could be at least about one mile apart, e.g., at least about 10 miles apart, e.g., at least about 100 miles apart. For example, remote terminal 170 could be in the same city as display device 120, remote terminal 170 could be in a different city than display device 120, remote terminal 170 could be in the same state as display device 120, remote terminal 170 could be in a different state than display device 120, remote terminal 170 could be in the same country as display device 120, or remote terminal 170 could be in a different country than display device 120, for example.

In certain embodiments, a separate, optional data communication/processing device such as data processing module 160 may be provided in analyte monitoring system 100. Data processing module 160 may include components to communicate using one or more wireless communication protocols such as, for example, but not limited to, infrared (IR) protocol, Bluetooth protocol, Zigbee protocol, and 802.11 wireless LAN protocol. Additional description of communication protocols including those based on Bluetooth protocol and/or Zigbee protocol can be found in U.S. Patent Publication No. 2006/0193375 incorporated herein by reference for all purposes. Data processing module 160 may further include communication ports, drivers or connectors to establish wired communication with one or more of display device 120, on body electronics 102, or remote terminal 170 including, for example, but not limited to USB connector and/or USB port, Ethernet connector and/or port, FireWire connector and/or port, or RS-232 port and/or connector.

In certain embodiments, data processing module 160 is programmed to transmit a polling or query signal to on body electronics 102 at a predetermined time interval (e.g., once every minute, once every five minutes, or the like), and in response, receive the monitored analyte level information from on body electronics 102. Data processing module 160 stores in its memory the received analyte level information, and/or relays or retransmits the received information to another device such as display device 120. More specifically in certain embodiments, data processing module 160 may be configured as a data relay device to retransmit or pass through the received analyte level data from on body electronics 102 to display device 120 or a remote terminal (for example, over a data network such as a cellular or WiFi data network) or both.

In certain embodiments, on body electronics 102 and data processing module 160 may be positioned on the skin surface of the user within a predetermined distance of each other (for example, about 1-12 inches, or about 1-10 inches, or about 1-7 inches, or about 1-5 inches) such that periodic communication between on body electronics 102 and data processing module 160 is maintained. Alternatively, data processing module 160 may be worn on a belt or clothing item of the user, such that the desired distance for communication between the on body electronics 102 and data processing module 160 for data communication is maintained. In a further aspect, the housing of data processing module 160 may be configured to couple to or engage with on body electronics 102 such that the two devices are combined or integrated as a single assembly and positioned on the skin surface. In further embodiments, data processing module 160 is detachably engaged or connected to on body electronics 102 providing additional modularity such that data processing module 160 may be optionally removed or reattached as desired.

Referring to FIG. 1, in certain embodiments, data processing module 160 is programmed to transmit a command or signal to on body electronics 102 at a predetermined time interval such as once every minute, or once every 5 minutes or once every 30 minutes or any other suitable or desired programmable time interval to request analyte related data from on body electronics 102. When data processing module 160 receives the requested analyte related data, it stores the received data. In this manner, analyte monitoring system 100 may be configured to receive the continuously monitored analyte related information at the programmed or programmable time interval, which is stored and/or displayed to the user. The stored data in data processing module 160 may be subsequently provided or transmitted to display device 120, remote terminal 170 or the like for subsequent data analysis such as identifying frequency of periods of glycemic level excursions over the monitored time period, or the frequency of the alarm event occurrence during the monitored time period, for example, to improve therapy related decisions. Using this information, the doctor, healthcare provider or the user may adjust or recommend modification to the diet, daily habits and routines such as exercise, and the like.

In another embodiment, data processing module 160 transmits a command or signal to on body electronics 102 to receive the analyte related data in response to a user activation of a switch provided on data processing module 160 or a user initiated command received from display device 120. In further embodiments, data processing module 160 is configured to transmit a command or signal to on body electronics 102 in response to receiving a user initiated command only after a predetermined time interval has elapsed. For example, in certain embodiments, if the user does not initiate communication within a programmed time period, such as, for example about 5 hours from last communication (or 10 hours from the last communication, or 24 hours from the last communication), the data processing module 160 may be programmed to automatically transmit a request command or signal to on body electronics 102. Alternatively, data processing module 160 may be programmed to activate an alarm to notify the user that a predetermined time period of time has elapsed since the last communication between the data processing module 160 and on body electronics 102. In this manner, users or healthcare providers may program or configure data processing module 160 to provide certain compliance with analyte monitoring regimen, so that frequent determination of analyte levels is maintained or performed by the user.

In certain embodiments, when a programmed or programmable alarm condition is detected (for example, a detected glucose level monitored by analyte sensor 302 that is outside a predetermined acceptable range indicating a physiological condition which requires attention or intervention for medical treatment or analysis (for example, a hypoglycemic condition, a hyperglycemic condition, an impending hyperglycemic condition or an impending hypoglycemic condition), the one or more output indications may be generated by the control logic or processor of the on body electronics 102 and output to the user on a user interface of on body electronics 102 so that corrective action may be timely taken. In addition to or alternatively, if display device 120 is within communication range, the output indications or alarm data may be communicated to display device 120 whose processor, upon detection of the alarm data reception, controls the display 122 to output one or more notification.

In certain embodiments, control logic or microprocessors of on body electronics 102 include software programs to determine future or anticipated analyte levels based on information obtained from analyte sensor 302, e.g., the current analyte level, the rate of change of the analyte level, the acceleration of the analyte level change, and/or analyte trend information determined based on stored monitored analyte data providing a historical trend or direction of analyte level fluctuation as function time during monitored time period. Predictive alarm parameters may be programmed or programmable in display device 120, or the on body electronics 102, or both, and output to the user in advance of anticipating the user's analyte level reaching the future level. This provides the user an opportunity to take timely corrective action.

Information, such as variation or fluctuation of the monitored analyte level as a function of time over the monitored time period providing analyte trend information, for example, may be determined by one or more control logic or microprocessors of display device 120, data processing module 160, and/or remote terminal 170, and/or on body electronics 102. Such information may be displayed as, for example, a graph (such as a line graph) to indicate to the user the current and/or historical and/or and predicted future analyte levels as measured and predicted by the analyte monitoring system 100. Such information may also be displayed as directional arrows (for example, see trend or directional arrow display 131) or other icon(s), e.g., the position of which on the screen relative to a reference point indicated whether the analyte level is increasing or decreasing as well as the acceleration or deceleration of the increase or decrease in analyte level. This information may be utilized by the user to determine any necessary corrective actions to ensure the analyte level remains within an acceptable and/or clinically safe range. Other visual indicators, including colors, flashing, fading, etc., as well as audio indicators including a change in pitch, volume, or tone of an audio output and/or vibratory or other tactile indicators may also be incorporated into the display of trend data as means of notifying the user of the current level and/or direction and/or rate of change of the monitored analyte level. For example, based on a determined rate of glucose change, programmed clinically significant glucose threshold levels (e.g., hyperglycemic and/or hypoglycemic levels), and current analyte level derived by an in vivo analyte sensor, the system 100 may include an algorithm stored on computer readable medium to determine the time it will take to reach a clinically significant level and will output notification in advance of reaching the clinically significant level, e.g., 30 minutes before a clinically significant level is anticipated, and/or 20 minutes, and/or 10 minutes, and/or 5 minutes, and/or 3 minutes, and/or 1 minute, and so on, with outputs increasing in intensity or the like.

Referring to FIG. 1, in certain embodiments, software algorithm(s) for execution by data processing module 160 may be stored in an external memory device such as an SD card, microSD card, compact flash card, XD card, Memory Stick card, Memory Stick Duo card, or USB memory stick/device including executable programs stored in such devices for execution upon connection to the respective one or more of the on body electronics 102, remote terminal 170 or display device 120. In a further aspect, software algorithms for execution by data processing module 160 may be provided to a communication device such as a mobile telephone including, for example, WiFi or Internet enabled smart phones or personal digital assistants (PDAs) as a downloadable application for execution by the downloading communication device.

Examples of smart phones include Windows®, Android™, iPhone® operating system, Palm® WebOS™, Blackberry® operating system, or Symbian® operating system based mobile telephones with data network connectivity functionality for data communication over an internet connection and/or a local area network (LAN). PDAs as described above include, for example, portable electronic devices including one or more microprocessors and data communication capability with a user interface (e.g., display/output unit and/or input unit, and configured for performing data processing, data upload/download over the internet, for example. In such embodiments, remote terminal 170 may be configured to provide the executable application software to the one or more of the communication devices described above when communication between the remote terminal 170 and the devices are established.

In still further embodiments, executable software applications may be provided over-the-air (OTA) as an OTA download such that wired connection to remote terminal 170 is not necessary. For example, executable applications may be automatically downloaded as software download to the communication device, and depending upon the configuration of the communication device, installed on the device for use automatically, or based on user confirmation or acknowledgement on the communication device to execute the installation of the application. The OTA download and installation of software may include software applications and/or routines that are updates or upgrades to the existing functions or features of data processing module 160 and/or display device 120.

Referring to remote terminal 170 of FIG. 1, in certain embodiments, new software and/or software updates such as software patches or fixes, firmware updates or software driver upgrades, among others, for display device 120 and/or on body electronics 102 and/or data processing module 160 may be provided by remote terminal 170 when communication between the remote terminal 170 and display device 120 and/or data processing module 160 is established. For example, software upgrades, executable programming changes or modification for on body electronics 102 may be received from remote terminal 170 by one or more of display device 120 or data processing module 160, and thereafter, provided to on body electronics 102 to update its software or programmable functions. For example, in certain embodiments, software received and installed in on body electronics 102 may include software bug fixes, modification to the previously stalled software parameters (modification to analyte related data storage time interval, resetting or adjusting time base or information of on body electronics 102, modification to the transmitted data type, data transmission sequence, or data storage time period, among others). Additional details describing field upgradability of software of portable electronic devices, and data processing are provided in U.S. application Ser. Nos. 12/698,124, 12/794,721, 12/699,653, and 12/699,844, and U.S. Provisional Application Nos. 61,359,265, and 61/325,155 the disclosure of which is incorporated by reference herein for all purposes.

### Exemplary Embodiment of the Inserter Assembly

In accordance with one embodiment of the invention, a sensor is positioned at least partially under the skin of a user by an inserter to measure analyte levels or concentrations, for example, glucose. As illustrated in FIG. 2, the inserter assembly 202 is positioned in an initial position with respect to mount 204 to insert sensor 302 (not shown in FIG. 2).

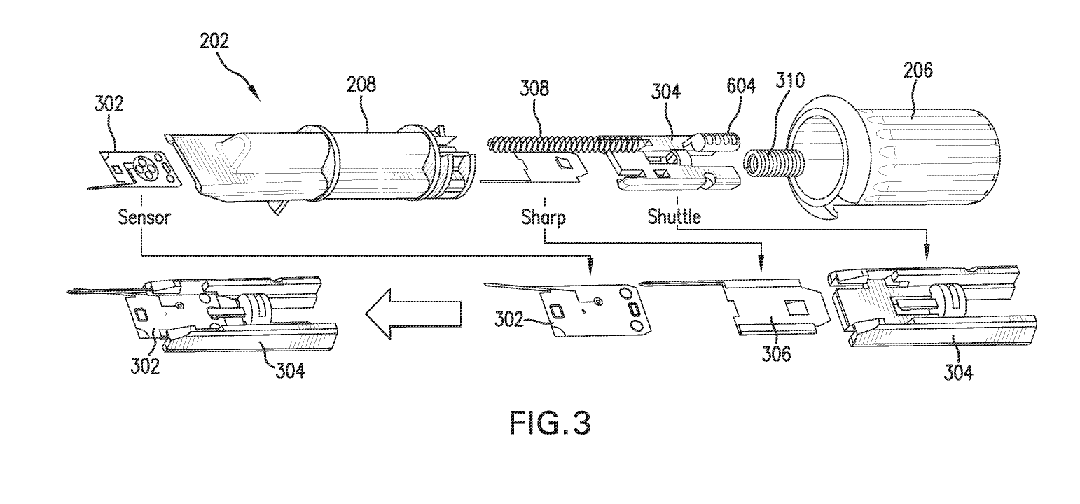

The inserter assembly 202, which can be preloaded with the sensor, is employed to insert the sensor through the skin of a user. Generally, as illustrated in FIG. 3, the inserter assembly 202 includes a rotatable trigger 206, first driver member 310, shuttle 304, second driver means 308, introducer sharp 306, and tubular housing 208.

In some embodiments, the rotatable trigger 206 is engaged to shuttle 304. A first driver member 310 is disposed between the rotatable trigger 206 and the shuttle 310. The shuttle is connected to an medical device to be inserted into a subject of a user, such as sensor 302. First driver member 310 can be configured to travel along a linear path, which includes the insertion path and retraction path. In this manner, as first driver member 310 moves along its linear path, shuttle 304 coupled to the first driver member also moves in a linear direction. The linear path of shuttle 304 includes an insertion direction, insertion point, retraction direction, and retraction point. Accordingly, at the insertion point of the linear path, the object to be inserted into the subject is released from shuttle 304.

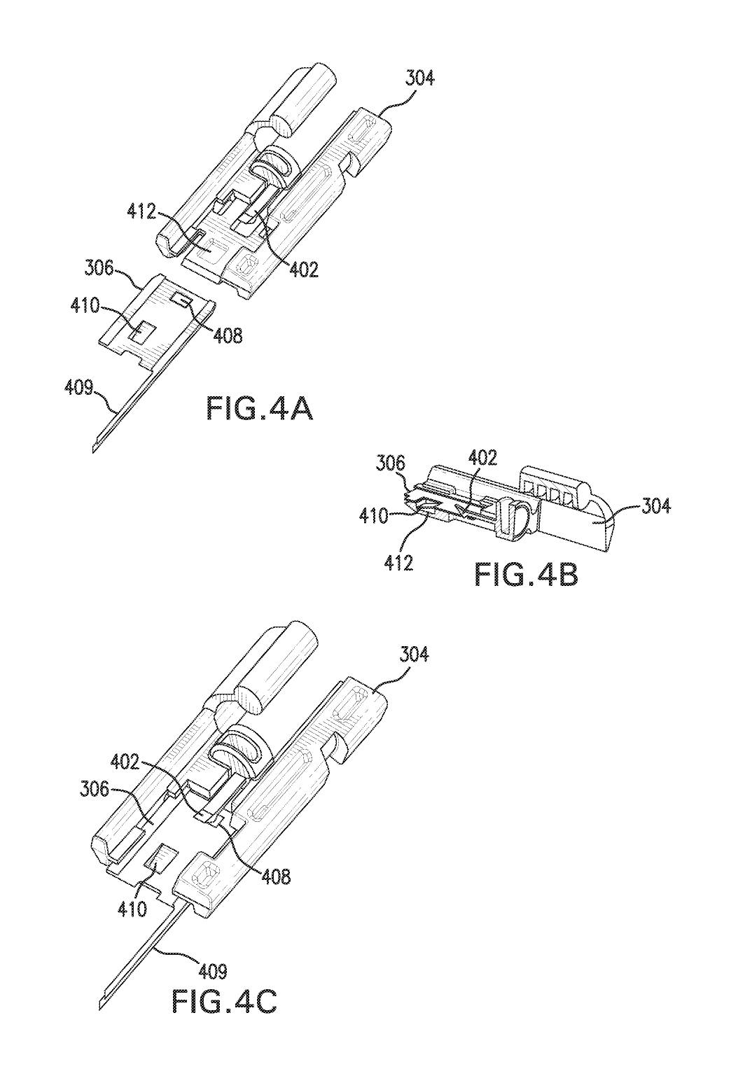

Referring now to FIGS. 4A-4C, in some embodiments, shuttle 304 can include an attachment structure, such as a first cantilever 402 (FIG. 4A) to engage a complimentary attachment structure disposed on introducer sharp 410. For example but not limitation, the shuttle can further include a second attachment structure such as a recess or a second cantilever 412 to engage a complementary second attachment structure disposed on introducer sharp 410. For example, the introducer sharp can be configured to define an aperture 408 for engagement with the attachment structure, e.g., first cantilever 402. Introducer sharp 306 can also be configured with a protrusion 410 to engage depression 412 disposed on the shuttle body. When shuttle 304 and introducer sharp 306 are brought into contact as shown in FIGS. 4B and 4C, first cantilever 402 can engage hole 408 and second cantilever 412 engages recess 410. Accordingly, shuttle 304 can be secured to introducer sharp 306 by one or more attachment structures to prevent their disengagement during the operation of inserter 202 or during shipping of inserter assembly.

In some embodiments, shuttle 304 may be molded overhangs which confine sharp 306 to a linear path as the sharp is assembled onto the shuttle. As sharp 306 reaches its assembled position, sharp finger 410 is released into the shuttle pocket 412. In this way, the sharp 306 is fully constrained and located on the shuttle 304.

Various other methods, however, can be employed to attach shuttle 304 to introducer sharp 306. For example, an adhesive or bonding agent can be used to attach shuttle 304 to introducer sharp 306.

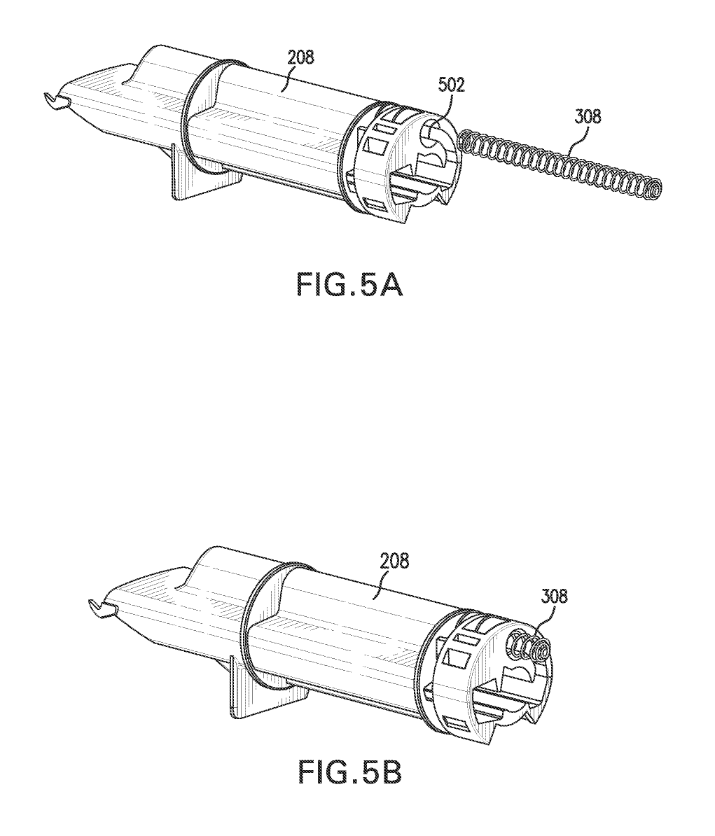

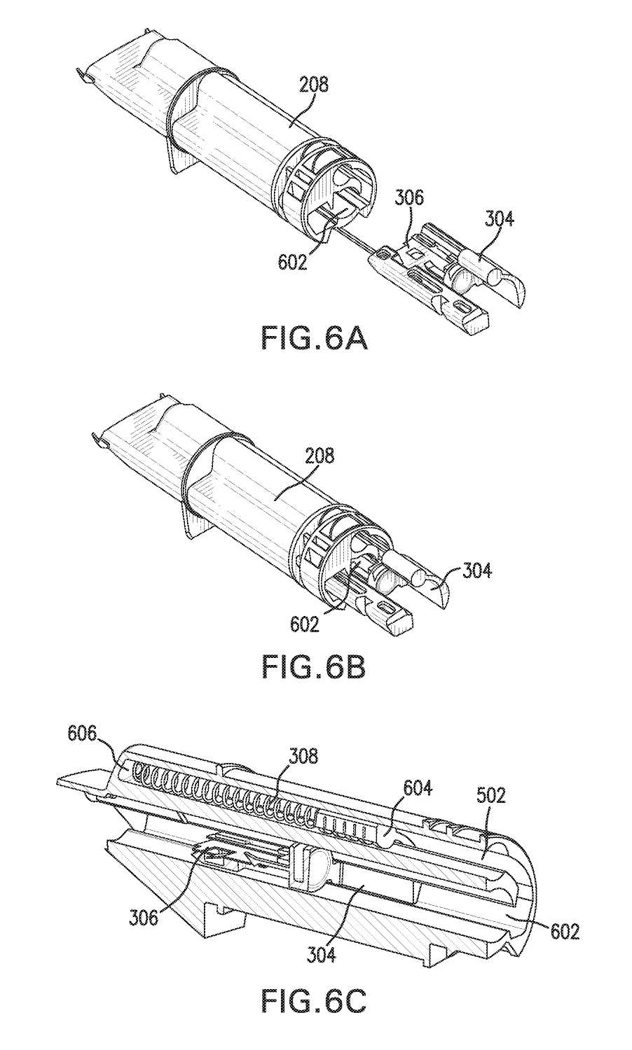

In some embodiments, a second driver member 308 is disposed in a channel 502 formed in the tubular housing 208, as depicted in FIGS. 5A and 5B. In some embodiments, the second driver member includes a spring, compression spring, torsion drive spring, constant force spring, clock spring, rolled sheet metal, elastic member, or motor, and the like, which can be disposed in the housing. After disposition of second driver member 308 in the channel 502, as shown in FIGS. 6A and 6B, shuttle 304 attached to introducer sharp 306 is disposed in the second channel 602 in communication with channel 502 of tubular housing 208. Shuttle 304 includes a flange 604 extending from a lateral side of shuttle body 304, as best seen in FIG. 3. When shuttle 304 is disposed in the second channel 610 of tubular housing 208, flange 604 abuts a proximal end of second driver member 308, e.g., retraction spring, disposed in the first channel 502. In this regard, the second driver member is confined in channel 502 between a closed end 606 of channel 502 and flange 604, as shown in FIG. 6C. The second driver member is configured to move along a linear path in the retraction direction after insertion of sensor 302. In this manner, the retraction of the second driver member pushes the shuttle (with attached introducer sharp 306) in the retraction direction by way of the contact of the proximal end of the driver member and the flange 604, until introducer sharp 306 reaches the retraction position within the trigger.

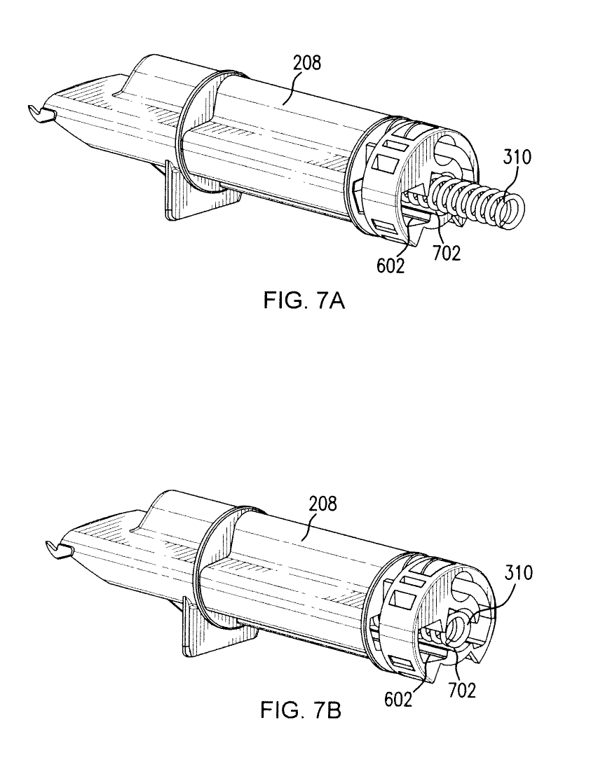

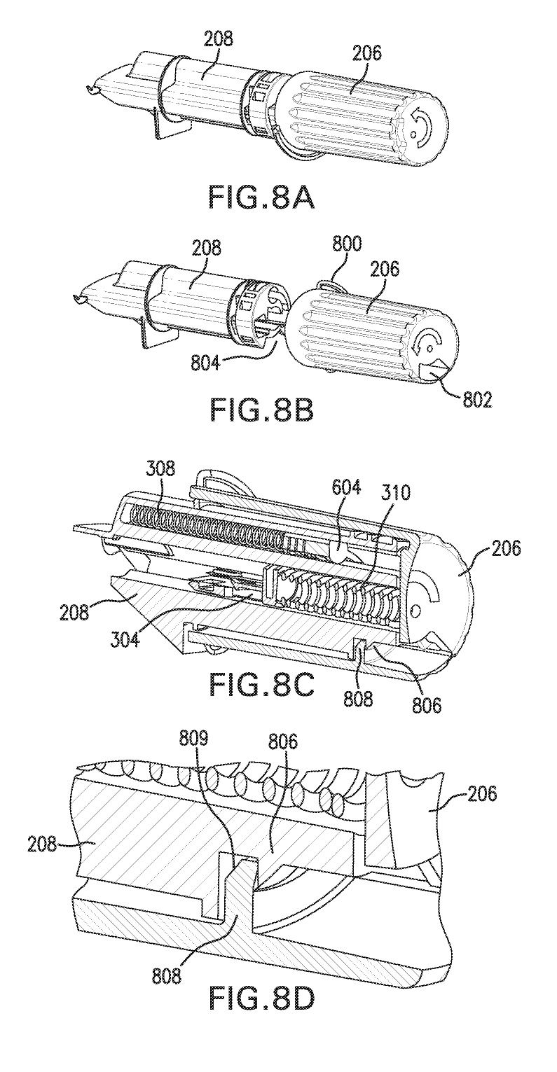

A first driver member 310 can be disposed in a third channel 702 of tubular housing 208, as shown in FIGS. 7A and 7B. In some embodiments, the third channel 702 is in communication with the first and second channels. Rotatable trigger 206 can then be placed proximate to the tubular housing 208, as depicted in FIG. 8A. Rotatable trigger 206 can include a window 802 which is aligned with depression 804 of housing 208 as shown in FIG. 8B. This allows rotatable trigger 206 to slide over tubular housing 208.

As shown in FIGS. 8C and 8D, rotatable trigger 206 includes a flange 808 disposed circumferentially about at least a portion of the inner wall of the rotatable trigger body. The flange 808 engages a groove 809 disposed circumferentially about the outer surface of tubular housing 208. In some embodiments, the flange 808 and groove 809 can be press fit until snap 806 engages projection 808. In this manner, tubular housing 208 is secured and is at least partially disposed within the body of rotatable trigger 206, and firing the inserter is impeded until disengagement of the flange and groove. Further, first drive member 310 is also be secured between the proximal end of shuttle 304 and proximal end of rotatable trigger 206, as can be seen in FIG. 8C.

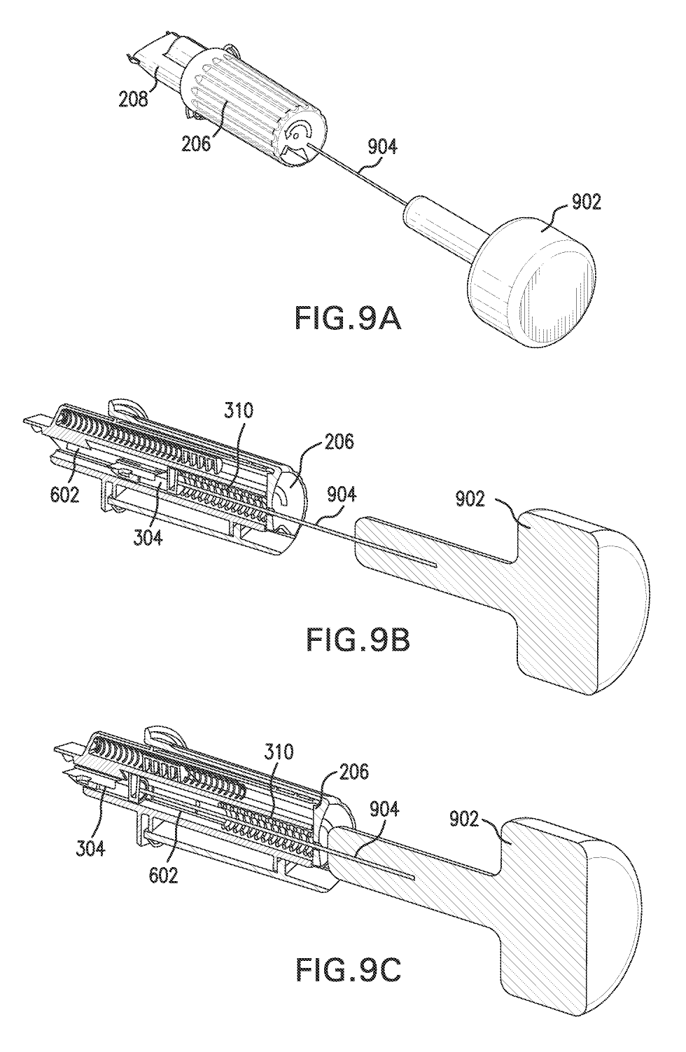

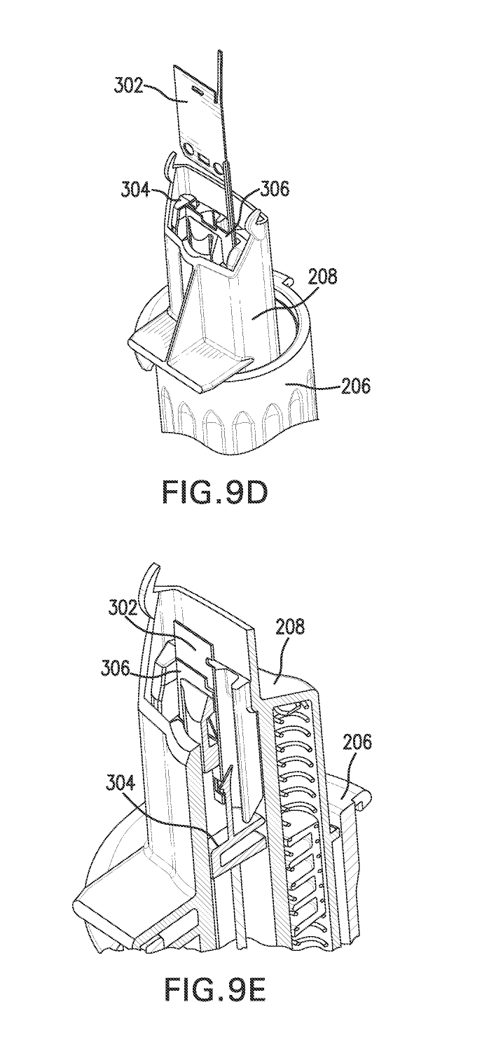

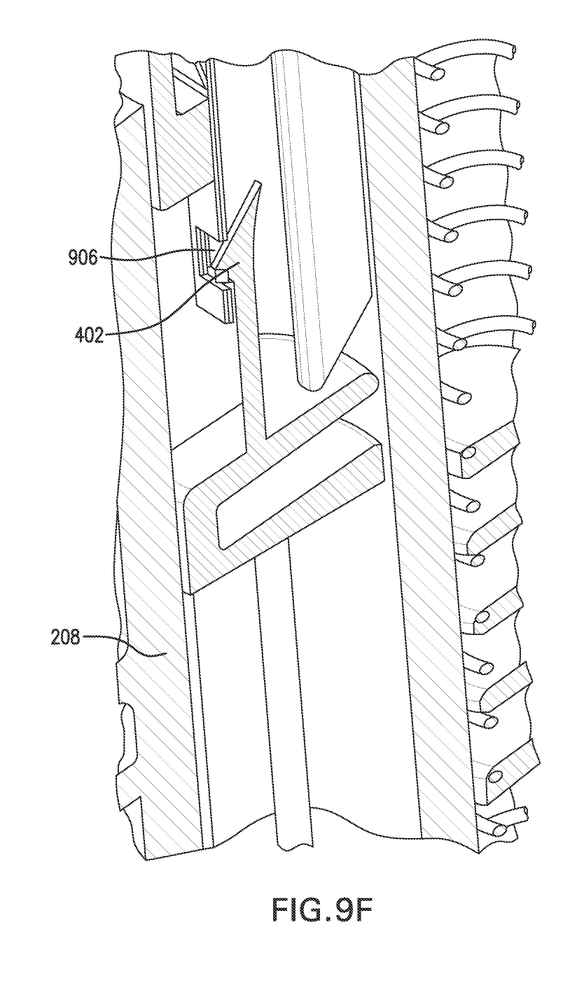

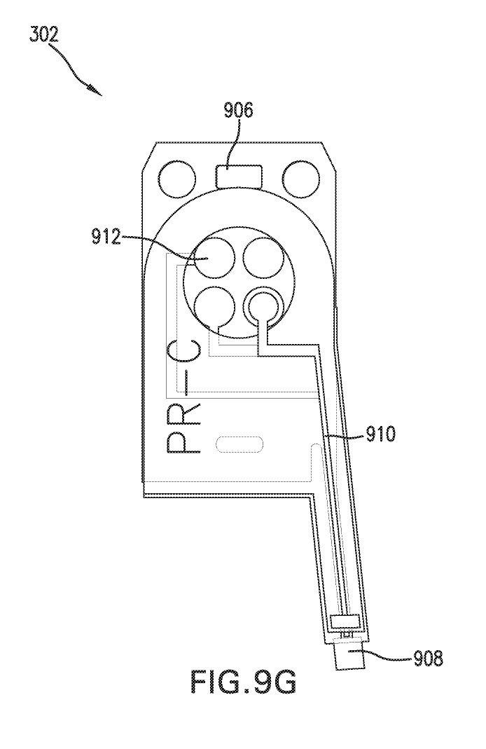

As described, the inserter assembly can be pre-loaded with a sensor. In some embodiments, a sensor loader 902 can be employed to attach the sensor 302 to introducer sharp 306, as shown in FIGS. 9A-9F. In accordance with one embodiment, an elongate projection 904 can be inserted into rotatable trigger 206 by a through hole disposed at the proximal end of the trigger body. The elongate protrusion 904 has a length sufficient to travel through a length of the tubular housing to contact and abut the proximal end of the shuttle 304. The sensor loader 902 can apply a force upon shuttle 304 to advance the shuttle 304 through channel 602, as shown in FIG. 9C. The elongate projection 904 can advance shuttle 304 and introducer sharp 306 downwardly until at least introducer sharp 302 extends distally from the distal end of the tubular housing 208, as shown in FIG. 9D. Sensor 302 can then be inserted into introducer sharp 302, as shown in FIGS. 9E and 9F. In some embodiments, sensor 302 is retained on the introducer sharp by a dimple formed in the sensor body. The dimple can form an interference fit with the introducer body. In other embodiments, sensor 302 includes a depression or hole 906 engaged to an attachment member, e.g., first cantilever 402 as the sensor is slidingly engaged to the introducer sharp. In this regard, the lateral sides of the introducer sharp can include flanges to receive the lateral edges of the sensor body. Sensor loader 902 can be removed from channel 602 after sensor 302 has been loaded.

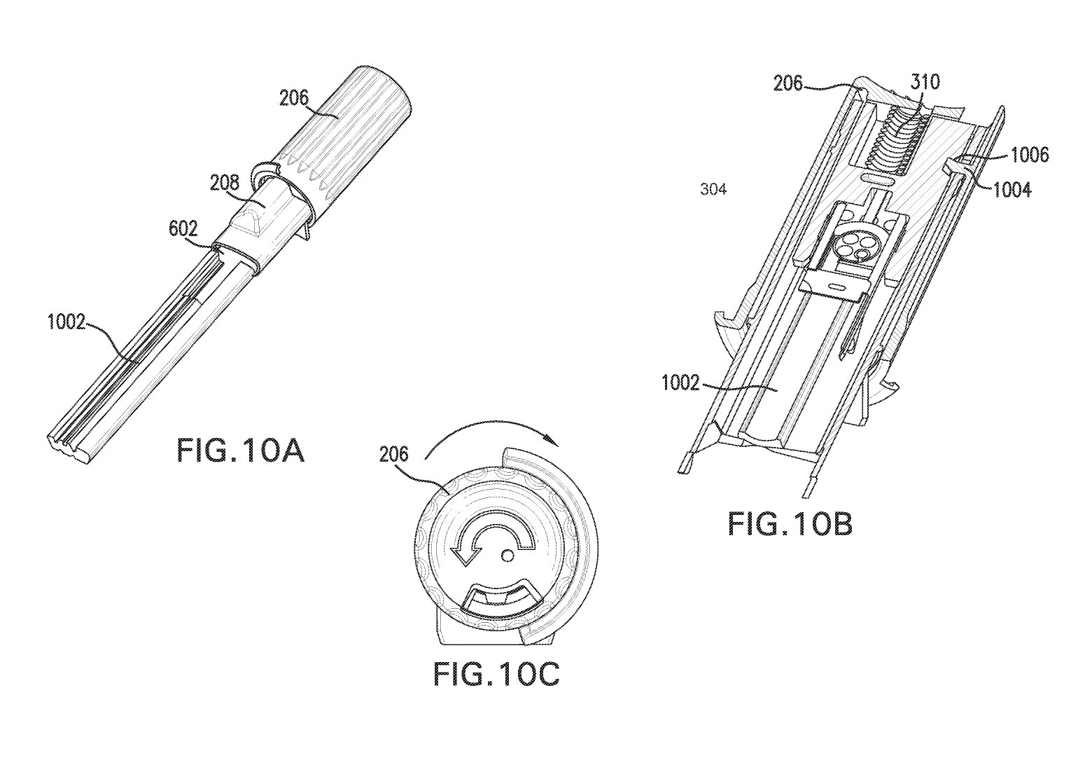

In another aspect, arming tool 1002 can be used to arm the inserter assembly. In this manner, arming tool 1002 can advance the shuttle 304 into channel 602 of tubular housing 208. By applying force to arming tool 1002, shuttle 304 is advanced until it abuts the end of the rotatable trigger 206 as shown in FIG. 10B. This also causes driver spring 310 to be compressed between rotatable trigger 206 and shuttle 304.

Rotatable trigger 206 can then be rotated in a first direction about an axis extending longitudinally from the distal end to the proximal end of the inserter, e.g., clockwise when viewed from the proximal end of inserter (as shown in FIG. 10C) until projection 1004, located on rotatable trigger 206, aligns with notch 1006. Thereafter, arming tool 1002 can be removed from shaft 602, leaving inserter 104 in an “armed” or “cocked position.” Rotation of rotatable trigger in a second direction, e.g., counterclockwise, causes disengagement of the projection 1004 and notch 1006. Disengagement allows first driver member 310 to longitudinally expand to drive and advance the shuttle and introducer sharp 306 to the insertion point. Retracting forces by retracting spring 308 withdraw sharp 306 to a withdrawal position.

### Mount

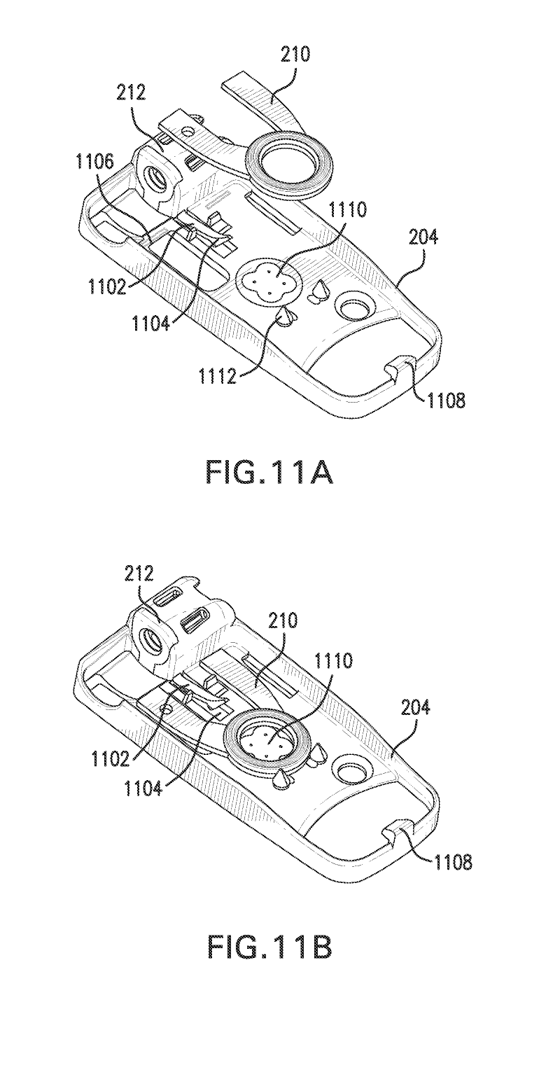

In some embodiments, inserter 202 can be affixed to mount 204 as depicted in FIG. 2. Mount 204, in some embodiments, as shown in FIG. 11A, includes a seal fixture 210 configured to protect a sensor disposed on the mount body at 1110. The seal fixture 210 can be affixed to the mount 204 at attachment points 1106. In this regard, seal fixture 210 can include first and second legs to attach to the mount. The seal fixture can further include a circular body to cover the sensor while positioned on the mount. The circular body can be attached to first and second legs. In some embodiments, the seal fixture 210 comprises elastomeric material and may be mechanically attached, welded, or glued to mount 204 (e.g., by laser welding,). FIG. 11B depicts mount 204 having seal fixture 210 attached. In some embodiments, the seal fixture 210 pivots upwardly and downwardly. In this manner, when sensor 302 is disposed on the upper surface of mount 204, the circular body of seal fixture 210 can cover sensor 302, and in particular, the sensor electric terminals or contacts. The pivoting action enables the seal fixture 210 to provide the inserter 202 with access to mount 204 for insertion of sensor 302.

Additionally, seal fixture 210 allows sensor 302 to be pressed flat to mount 204 (i.e., disposed in a horizontal orientation with respect to mount 202). This allows the height of the overall system to be minimized by allowing the horizontally positioned circuit board to make contact with sensor 302 in a horizontal orientation. Additionally, spikes 1112 on mount 204 align and position sensor 302 after the inserter is removed.

In some embodiments, mount 204 comprises electrical contacts 1110 to which the sensor is in communication when positioned on mount 204. The mount 204 can further include electric leads (not shown), which may be embedded in mount 204. In some embodiments, a plurality of electrodes are disposed on the sensor body. The electrodes may include a working electrode, counter electrode and reference electrode, disposed at the distal tip 908 of the sensor 302, as illustrated in FIG. 9G. The sensor 302 can further include conductive traces 910 extending from electrodes to corresponding respective contacts to define the sensor electronic circuitry. Thus, electrical communication can be established between the mount and the sensor by contacting the sensor electronics to the mount electrical contacts 1110.

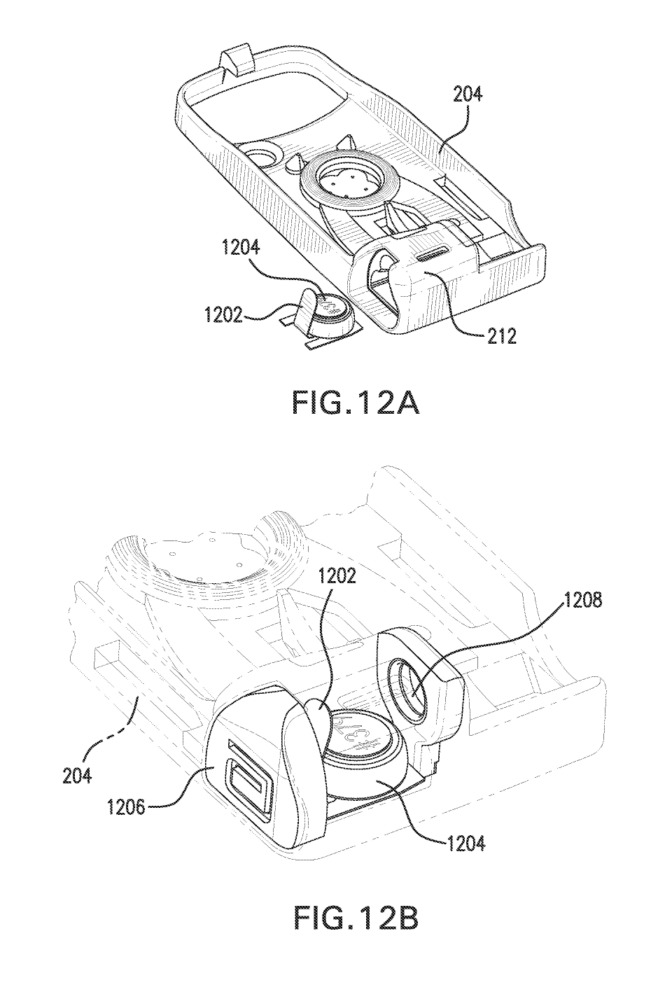

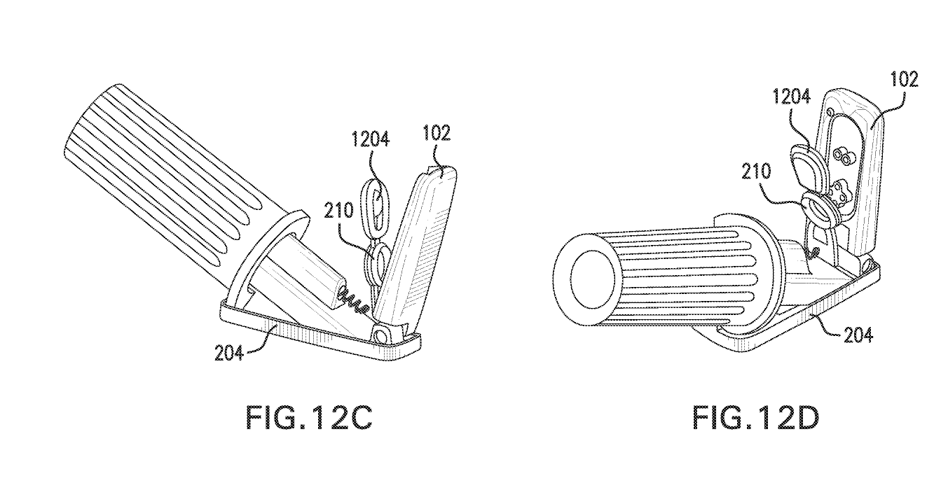

The mount can also comprise one or more latches, e.g., first latch 1102, second latch 1108, to engage electronics unit 102, such as a transmitter or transceiver component thereof. The electronics unit 102 can be configured to snap on to mount 204 or otherwise engage onto the mount. The mount 204 further includes a power compartment 212 to receive a power source, such as a battery. As shown in shown in FIGS. 12A and 12B, the power source 1204 can be received into the power compartment 212. In some embodiments, the battery 1204 can be placed on a battery holder 1202 and inserted into battery housing 212. Preferably, battery 1204 is a coin battery, such as an Energizer® 379 coin battery. Battery holder 1202 may be made of any conductive material, such as stainless steel. Battery holder 1202 provides power to sensor contacts 1110 through the aforementioned electrical leads.

In some embodiments, the power compartment can further include a door or closure 1206, such as a seal to enclose the compartment and contain the power source within the compartment, as show in FIG. 12B. Battery closure 1206 can either be permanently affixed to battery housing 212 or removeably attached. For example, battery closure 1206 may be permanently affixed utilizing ultrasonic welding techniques, after the power source is inserted into the compartment. Press-fitting or adhesive are alternate methods of securing battery closure 1206 to mount 204.

In another embodiment, battery 1204 may be attached to seal fixture 210 as shown in FIGS. 12C-12D instead of being located in battery housing 212. In this embodiment, rotation of on body electronics unit 102 seals and connects on body electronics unit 102 to battery 1204.

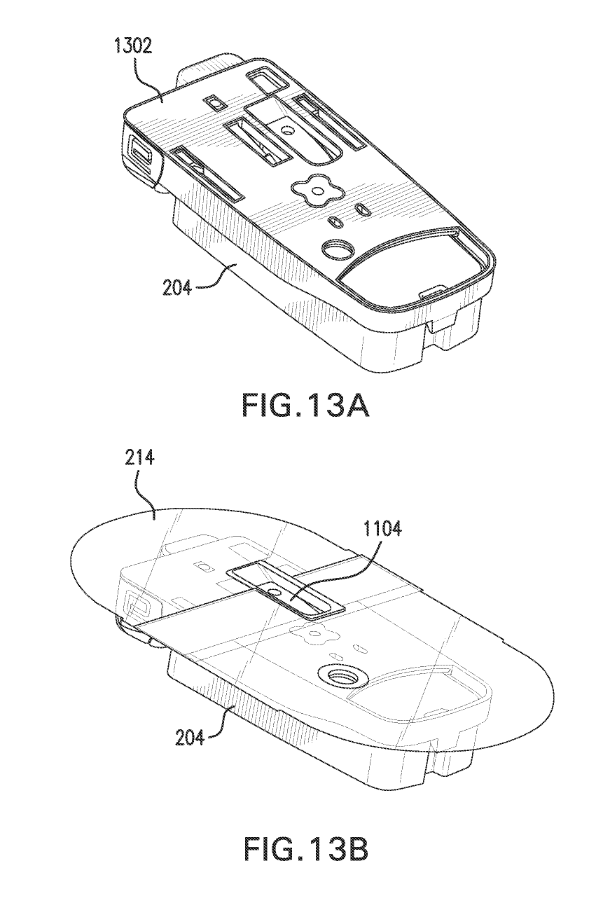

As described above, the mount 204 includes a surface adapted to attach to the user. Referring now to FIG. 13A, shown is mount 204 with skin adhesive assembly 1302 attached. In some embodiments, skin adhesive pad assembly 1302 is covered with an adhesive (e.g., using an acid brush to evenly coat the bonding surface). Adhesive pad 214 can then be affixed to adhesive pad assembly 1302. Both adhesive pad assembly 1302 and adhesive pad 214 can contain a hole positioned at sensor opening 1104 in mount 204 for allowing the passage of sensor 302 when it is inserted. In this manner, during use, a liner can be removed from the adhesive surface so the mount can be easily positioned on the body of the user.

### On Body Electronics Unit

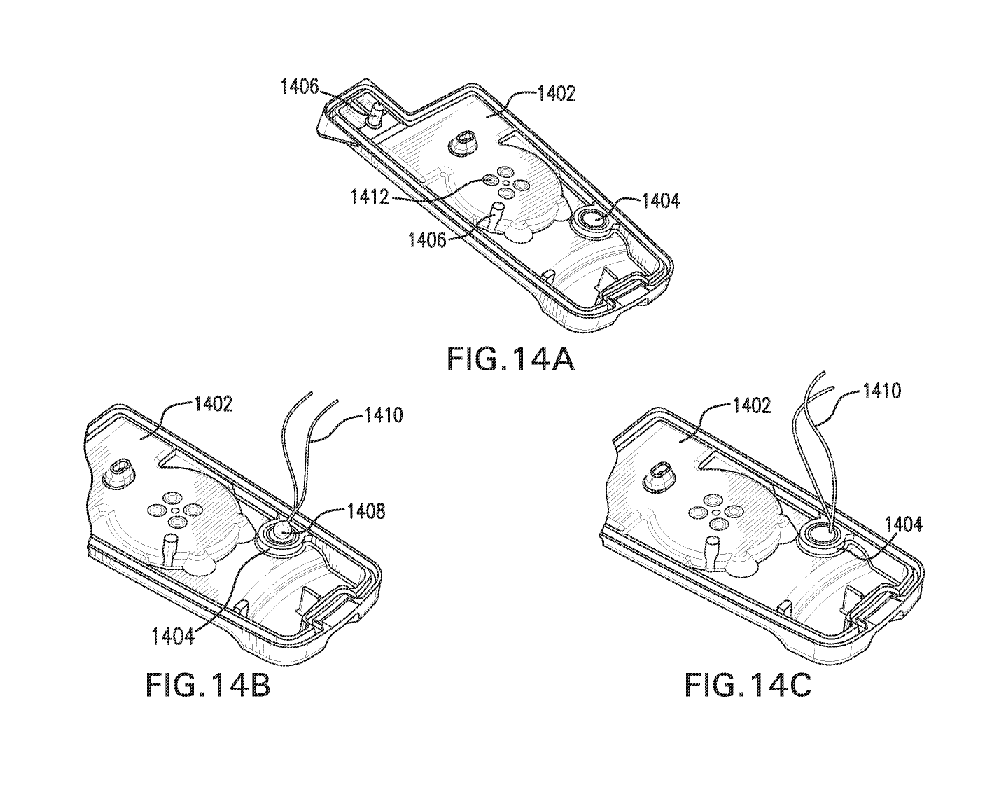

The assembly of one embodiment of on body electronics unit 102 is shown in FIGS. 14A-17D. Now with reference to FIG. 14A, the on body electronics unit 102 can include a base 1402. The base 1402 can comprise one or more conductive contacts 1412 disposed on the on body electronics unit. The conductive contacts 1412 are capable of forming an electrical communication with the sensor when the on body electronics unit is attached to the mount 204. The base 1402 can further include one or more posts 1406 extending upwardly from the base surface. The one or more posts 1406 can align and engage with recesses in a lid (not currently shown) and form the body of the on body electronics unit.

As shown in FIG. 14B, in some embodiments, the on body electronics unit includes one or more thermocouple openings 1404 configured to receive one or more thermocouples 1408. For example, thermocouple opening 1404 can be filled with a material that will affix thermocouple 1408 in thermocouple opening, such as epoxy compound, such as Master bond® Epoxy compound EP30AN.

The thermocouple 1408 includes one or more leads 1410 extending from the thermocouple 1404 at thermocouple opening 1404. In this manner, the on body electronics unit can be configured to determine on-skin temperature levels for use in the analyte estimation determination based on the signals received from the sensor. For example, a measured temperature reading can be obtained for each sampled signal from the sensor by the thermocouple 1408 disposed on the on body electronics unit. In some embodiments, a second temperature measurement can be obtained, such as an ambient temperature reading by employment of a second thermocouple 1408. In some embodiments, thermocouple 1408 is a Thermometric® MC65 thermocouple.

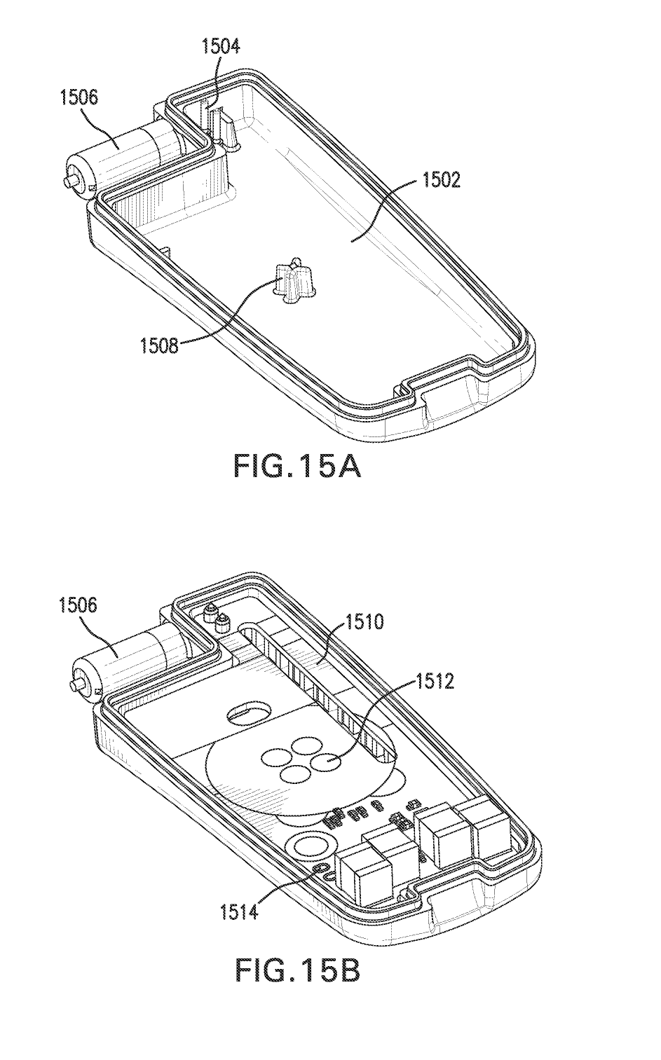

The lid 1502 of on body electronics unit 102, is shown in FIG. 15A. In one embodiment, lid 1502 can generally comprise one or more posts 1504 and support 1508. The lid 1502 can further include a printed circuit board 1510 disposed between the base and lid, as shown in FIG. 15B. As shown, printed circuit board 1510 comprises conductive contacts 1512, and can include thermocouple contact 1514.

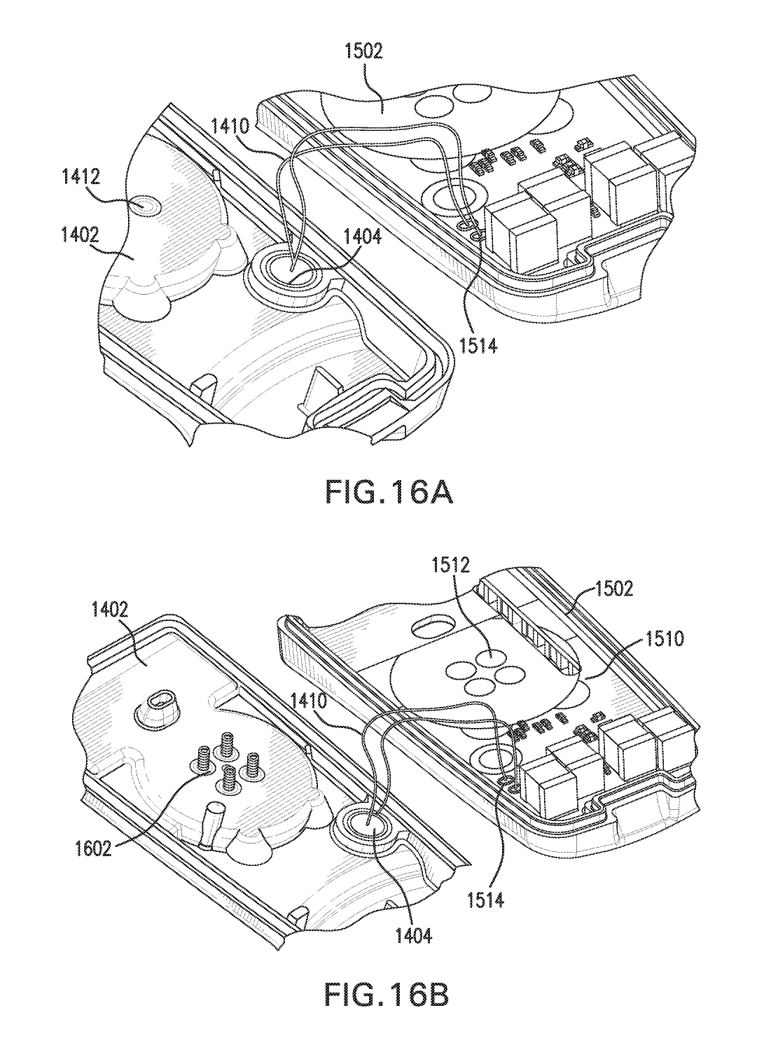

In some embodiments, the printed circuit board body 1510 is disposed on the support 1508, which can be configured to allow the printed circuit board 1510 to rest at a height above the base of lid 1502. Printed circuit board 1510 can include leads 1410 connected to thermocouple contacts 1514, as shown in FIG. 16A. Any connection technique, such as soldering, may be utilized. Additionally, contact springs 1602 can be placed on conductive contacts 1512 as shown in FIG. 16B. Contact springs 1602 allow for electrical communication between conductive contacts 1512 and 1412 when on body electronics unit 102 is assembled.

In certain embodiments, one or more application-specific integrated circuits (ASIC) may be used to implement one or more functions or routines associated with the operations of the data processing unit (and/or receiver unit) using for example one or more state machines and buffers. The electronics unit 102 illustrated in FIG. 1 may be equipped with sufficient memory to store the data of interest (such as analyte data) for extended periods of time ranging, e.g., from about one to about several data points to the number of data points obtained for an entire wear period of about 1 day to about several days, e.g., about one week to about several weeks or more, e.g., about one month to about several months or more.

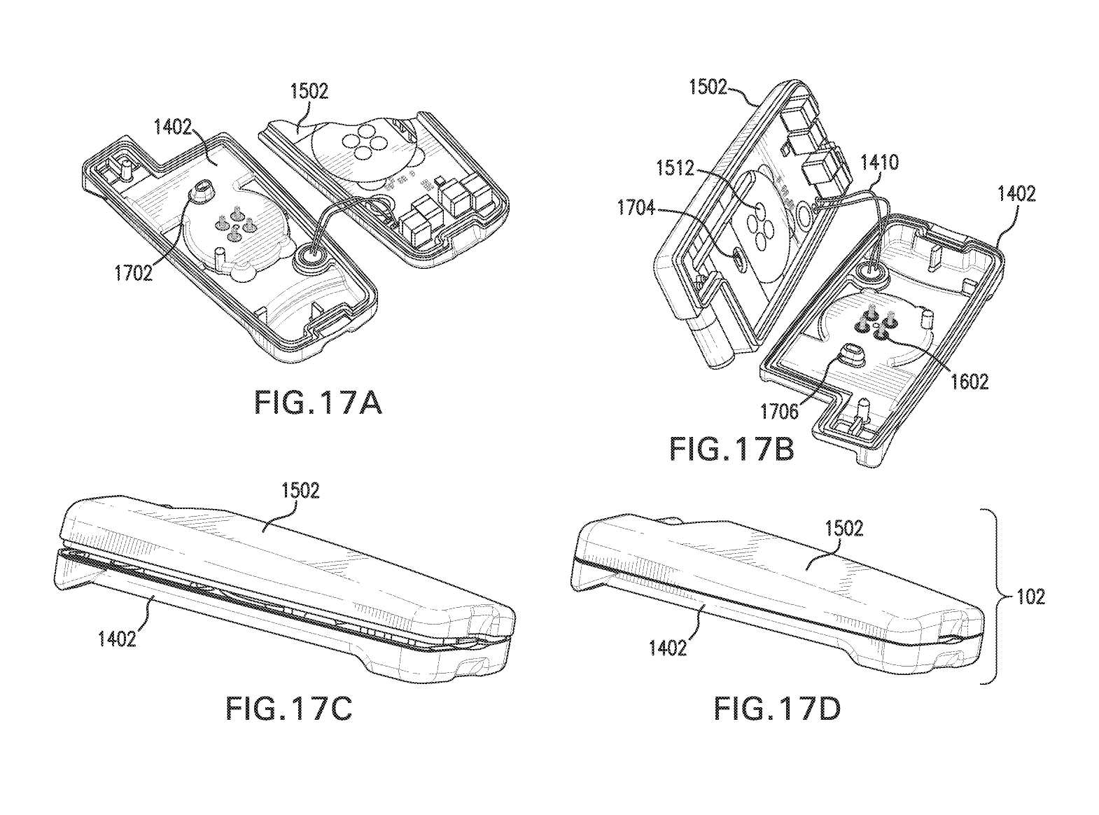

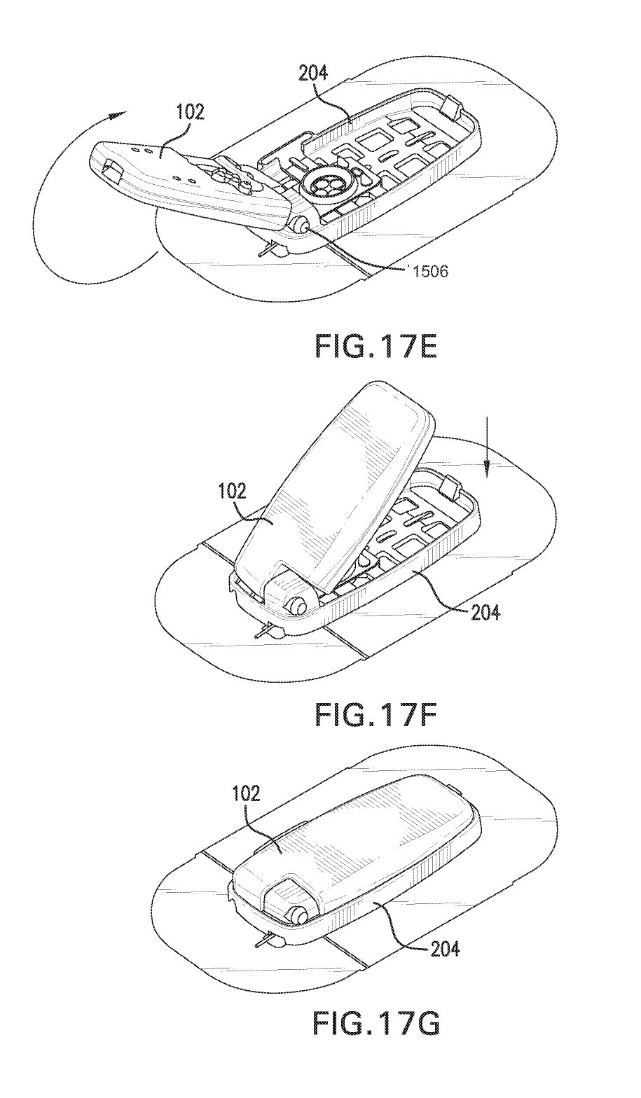

A bonding agent, such as an epoxy, can be placed in a bead around the outer perimeter of base 1402 as shown in FIG. 15A. In some embodiments, a bonding agent can also be applied around the perimeter of lid 1502. In this manner, base 1402 and lid 1502 can be joined to define on body electronics unit 102, as shown in FIGS. 17B-17D. As shown, various elements of base 1402 and lid 1502 line up when on body electronics unit 102 is assembled. For example, base 1402 contains post 1706 which aligns with recess 1704 when on body electronics unit 102 is assembled. On body electronics unit 102 is fully assembled when base 1402 and lid 1502 are bonded to each other as shown in FIG. 17D.

The body of the on body electronics unit, as illustrated in FIG. 17D includes a projection member 1506 (best seen in FIG. 15A). Projection member 1506 is configured to be received within the power compartment 212 of mount 204, as shown in FIG. 17E. In some embodiments, the projection member 1506 includes conductive contacts configured to establish electrical communication with the power source contained within power compartment 212 of mount 204. In this manner, the on body electronics unit 102 does not require its own internal power source to power up. Instead, the on body electronics unit can be powered by the power source contained on the mount 204. Accordingly, the on body electronics unit 102 can be configured to have a smaller configuration and a reduced profile because it can be constructed without the requirement for an on-board battery.

In some embodiments, the projection member 1506 is tubular. In this manner, the on body electronics unit can form a rotational engagement with the mount 204 and pivot in an upwardly and downwardly direction, as illustrated in FIGS. 17E to 17G. Further, power compartment 212 may contain a soft durometer material to form a seal around projection member 1508 as it is inserted. The durometer material may alternatively be located on projection member 1508.

### Sensor Assembly and Inserter Assembly Operation

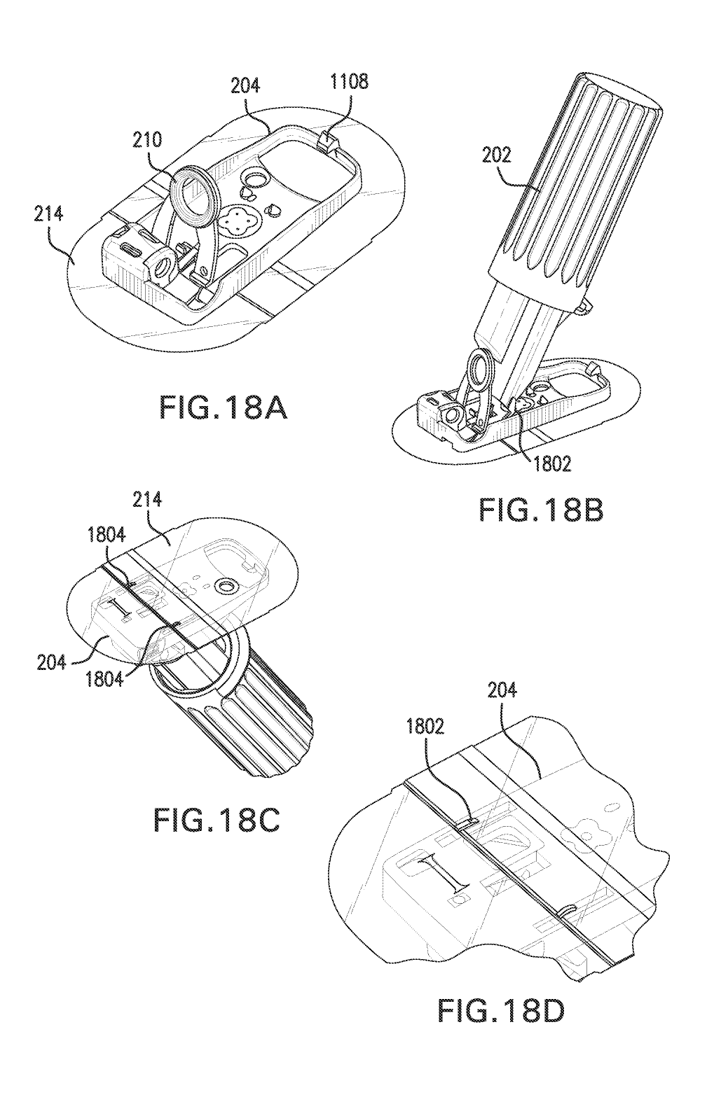

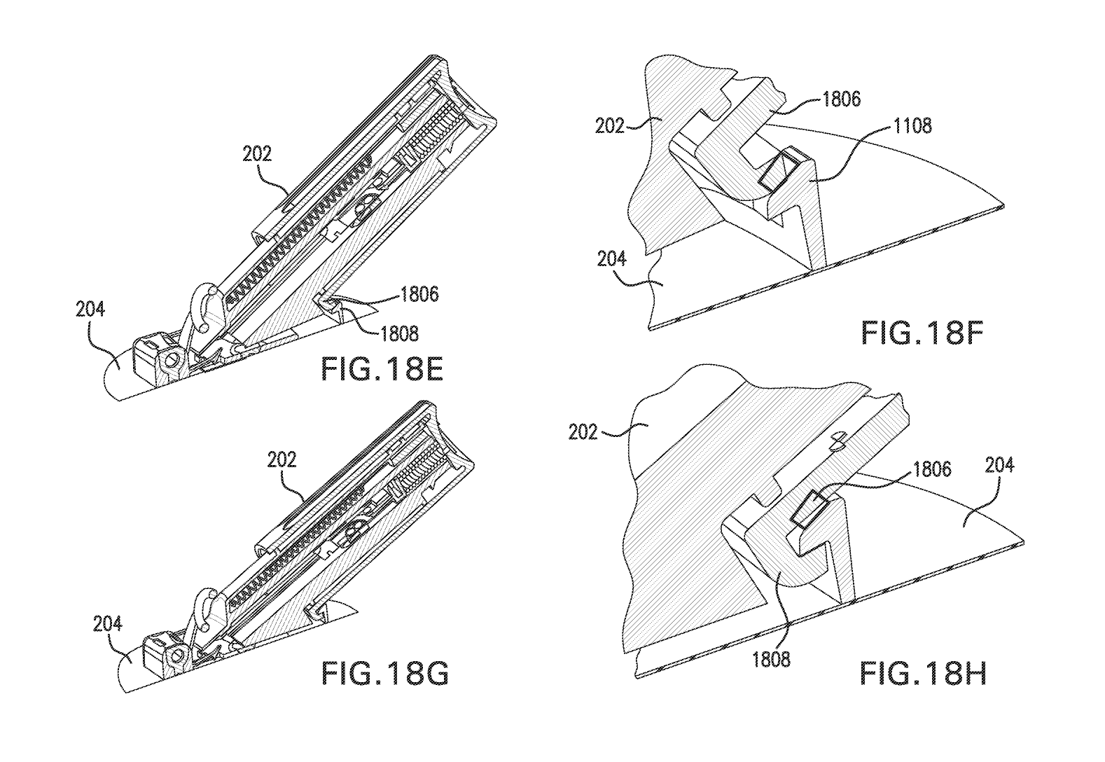

The fully assembled mount 204 is shown in FIG. 18A. Before inserter 202 can be attached to mount 204, seal fixture 210 must be lifted so that inserter 202 can be accommodated. In some embodiments, one or more hooks 1802 are disposed on inserter 202, and in particular the distal end of the tubular housing. The one or more hooks 1802 can be inserted into hook openings 1804 located on mount 204 (as depicted in FIGS. 18C-18D). Inserter 202 can then be pivoted down toward mount 204 by engagement of hooks 1802, as shown in FIG. 18E. As inserter 202 is rotated downward, hook 1806 approaches latch 1108 as shown in FIG. 18F. By applying a downward force on inserter 202, hook 1806 and latch 1108 can engage as shown in FIG. 18H, thereby affixing inserter 202 to mount 204. Inserter 202 can be removed from mount 204 by rotating rotatable trigger 206 so that window 802 (as best shown in FIG. 8B) aligns with latch 1108.

The engaged inserter 202 and mount 204 can be attached to the skin of a user (by way of the adhesive surface 214 of mount 204) at the desired location for implantation of the sensor. The rotatable trigger 206 is rotated to insert the sensor 302 into the skin. In this manner, projection 1004 disengages from groove or notch 1006, thereby releasing driver member 310. The force exerted by driver member 310 drives shuttle 304 along channel 602 until introducer sharp 306 pierces the user's skin. An adhesive, located on sensor body 302 can exert a force once contacting the skin to assist displacement of the sensor from the shuttle 304. After which a second driver, such as a driver member 308, draws shuttle 304 in an opposite retraction direction through channel 306.

Alternatively, a latch (not shown), located on the mount 204, engages a window on the sensor 302 at the insertion depth. The latch holds the sensor 302 at the inserted position even as the sharp 304 retracts upward; the second driver, such as a driver member 308, draws shuttle 304 in an opposite retraction direction through channel 306.

In one embodiment, a stationary finger tab can be located on mount 204 or tubular housing 208. A similar tab can be placed on rotatable trigger 206 so that by squeezing the two tabs together, rotation of rotatable trigger 206 occurs. This feature can reduce stress on the bond between adhesive patch 214 and a user's skin as the resistance torque required to turn rotatable trigger 206 would not be supported solely by adhesive patch 206, but also by a user's actuation hand.

After deployment of sensor 302, inserter 202 can be then be removed from mount 204 by rotating the trigger. Seal fixture 210 can pivot downward to cover sensor 302 and maintain sensor contacts 912 of sensor 302 come into electrical contact with sensor contact 1110 of mount 204.

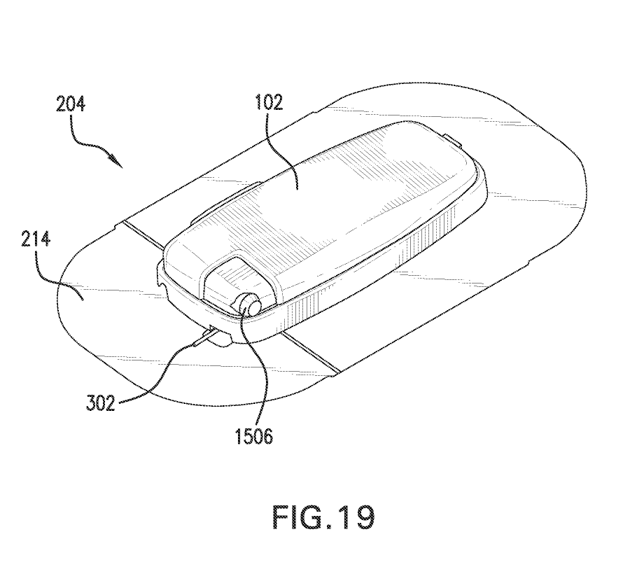

After inserter 202 is removed from mount 204, on body electronics unit 102 can be attached as shown in FIG. 19. First, projection 1506 is inserted through opening 1208 of power compartment to engage the on body electronics unit to the mount. Such engagement places battery holder 1202 into electrical contact with on body electronics unit 102. On body electronics unit 102 is thus in electrical contact with battery 1204. As described, this assembly allows the size of on body electronics unit 102 to be greatly reduced because it does not have to house a power supply.

By locating battery 1204 on mount 204, the size of mount 204 can also be greatly reduced because there is no need for a user accessible battery door and a battery seal inside the on body electronics unit. Additionally, the challenge to the user of handling a small battery is removed and the power requirement is reduced to one insertion/mount wear cycle per battery. Thus, battery replacement is made transparent to the user and the burden is removed from the user to remember to replace the battery of the on body electronics unit. Likewise, in a rechargeable on body electronics unit use scenario, the burden is removed from the user of having to keep different on body electronics units regularly charged.

In some embodiments, “sleep mode” of on body electronics unit 102 is automated, as power is only supplied to on body electronics unit 102 when it is inserted into mount 204. Additionally, the limited life of battery 1204 prevents extended use of sensor 302 past recommended wear.

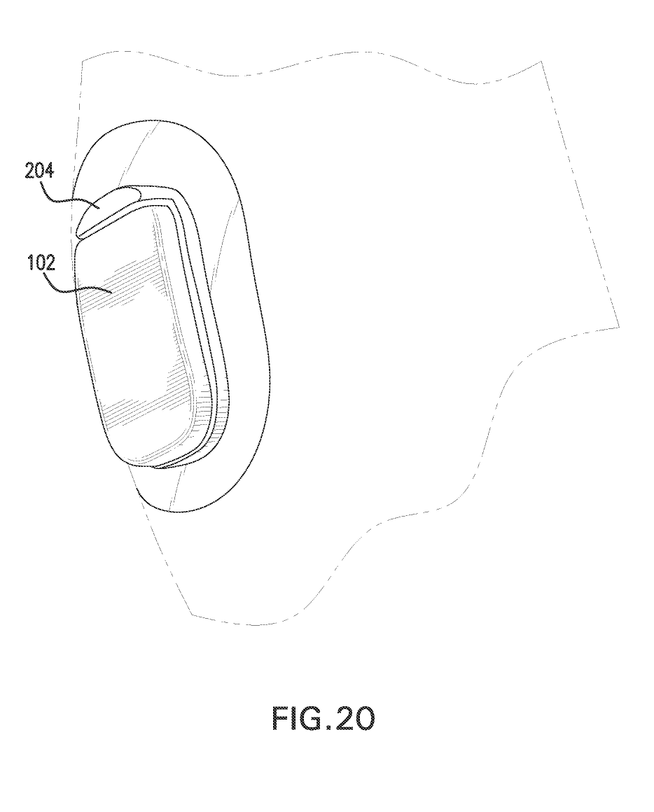

On body electronics unit 102 can be rotated toward mount 204 until latch 1108 engages recess 1708 (as shown in FIG. 12), thereby attaching on body electronics unit 102 to mount 204. Additionally, this connection can place sensor 302 into electrical contact with on body electronics unit 102 through contacts located on the on body electronics unit 102. Mount 204 and on body electronics unit 102 attached to a user is depicted in FIG. 20.

In another aspect, after the wear cycle of sensor 302 and mount 204 are completed, on body electronics unit 102 can be used on another sensor/mount assembly because it can receive power from a fresh power source located in mount 204. This allows all disposable components to be located on mount 204. This design is advantageous because the disposable materials (such as soft durometer materials), which are ideal to create seals with minimal force requirements, are materials which are more likely to wear out with repeated use. Because the disposable materials are located on mount 204, a user can be assured that all seals are in ideal condition before each use. Additionally, the wear and tear between harder plastic or metallic mating surface on body electronics unit 102 and mount 204 will be taken care of an disposed of on each mount.

### Sensor

In some embodiments, sensor 302 draws its power from a power source, such as a battery, located in power compartment 212 of mount 204 (as shown in FIG. 12B). Electric leads from compartment 212 can be used to connect the power source to sensor contacts 912 (as shown in FIG. 9G). In some embodiments, the leads can be integral to mount 204.

In some embodiments, sensor 302 comprises a substrate, one or more electrodes, a sensing layer and a barrier layer, as described below and disclosed in U.S. Pat. Nos. 6,284,478 and 6,990,366, the disclosures of which are incorporated herein by reference.

In some embodiments, the substrate is formed from a relatively flexible material. Suitable materials for a flexible substrate include, for example, non-conducting plastic or polymeric materials and other non-conducting, flexible, deformable materials. Suitable plastic or polymeric materials include thermoplastics such as polycarbonates, polyesters (e.g., Mylar® and polyethylene terephthalate (PET)), polyvinyl chloride (PVC), polyurethanes, polyethers, polyamides, polyimides, or copolymers of these thermoplastics, such as PETG (glycol-modified polyethylene terephthalate). In other embodiments, the sensor includes a relatively rigid substrate. Suitable examples of rigid materials that may be used to form the substrate include poorly conducting ceramics, such as aluminum oxide and silicon dioxide. Further, the substrate can be formed from an insulating material. Suitable insulating materials include polyurethane, Teflon (fluorinated polymers), polyethyleneterephthalate (PET, Dacron) or polyimide.

Sensor 302 can include a distal end and a proximal end having different widths. In such embodiments, the distal end of the substrate may have a relatively narrow width (as best depicted in FIG. 9G). Moreover, sensors intended to be partially positioned into the tissue of a user's body can be configured to have narrow distal end or distal point to facilitate the insertion of the sensor. For example, for insertable sensors designed for continuous or periodic monitoring of the analyte during normal activities of the patient, a distal end of the sensor which is to be implanted into the user has a width of 2 mm or less, preferably 1 mm or less, and more preferably 0.5 mm or less.

A plurality of electrodes are disposed at the distal tip 908 of sensor 302. The electrodes can include working electrode, counter electrode and reference electrode. Other embodiments, however, can include less or more electrodes. For example, a two electrode sensor can be utilized. In some embodiments, the sensor is a self-powered analyte sensor, which is capable of spontaneously passing a currently directly proportional to analyte concentration in the absence of an external power source. Any exemplary sensor is described in U.S. application Ser. No. 12/393,921, filed Feb. 26, 2009, and entitled “Self-Powered Analyte Sensor,” which is hereby incorporated by reference in its entirety herein.

Each of the electrodes are formed from conductive material, for example, a non-corroding metal or carbon wire. Suitable conductive materials include, for example, vitreous carbon, graphite, silver, silver-chloride, platinum, palladium, or gold. The conductive material can be applied to the substrate by various techniques including laser ablation, printing, etching, and photolithography. In one embodiment, each of the electrodes are formed from gold by a laser ablation technique. As further illustrated, the sensor 302 includes conductive traces 910 extending from electrodes to corresponding, respective contacts 912 to define the sensor electronic circuitry. In one embodiment, an insulating substrate (e.g., dielectric material) and electrodes are arranged in a stacked orientation (i.e., insulating substrate disposed between electrodes). Alternatively, the electrodes can be arranged in a side by side orientation, as described in U.S. Pat. No. 6,175,752, the disclosure of which is incorporated herein by reference for all purposes.

Sensor 302 has a sensing layer including one or more components designed to facilitate the electrolysis of the analyte of interest. The components, for example, may be immobilized on the working electrode. Alternatively, the components of the sensing layer may be immobilized within or between one or more membranes or films disposed over the working electrode or the components may be immobilized in a polymeric or sol-gel matrix. Examples of immobilized sensing layers are described in U.S. Pat. Nos. 5,262,035, 5,264,104, 5,264,105, 5,320,725, 5,593,852, and 5,665,222, each of which is incorporated herein by reference for all purposes.

Various other modifications and alterations in the structure and method of operation of this invention will be apparent to those skilled in the art without departing from the scope and spirit of the invention. Although the invention has been described in connection with specific preferred embodiments, it should be understood that the invention as claimed should not be unduly limited to such specific embodiments. It is intended that the following claims define the scope of the present invention and that structures and methods within the scope of these claims Additional detailed description of embodiments of the disclosed subject matter are provided in but not limited to: U.S. Pat. No. 7,299,082; U.S. Pat. No. 7,167,818; U.S. Pat. No. 7,041,468; U.S. Pat. No. 6,942,518; U.S. Pat. No. 6,893,545; U.S. Pat. No. 6,881,551; U.S. Pat. No. 6,773,671; U.S. Pat. No. 6,764,581; U.S. Pat. No. 6,749,740; U.S. Pat. No. 6,746,582; U.S. Pat. No. 6,736,957; U.S. Pat. No. 6,730,200; U.S. Pat. No. 6,676,816; U.S. Pat. No. 6,618,934; U.S. Pat. No. 6,616,819; U.S. Pat. No. 6,600,997; U.S. Pat. No. 6,592,745; U.S. Pat. No. 6,591,125; U.S. Pat. No. 6,560,471; U.S. Pat. No. 6,540,891; U.S. Pat. No. 6,514,718; U.S. Pat. No. 6,514,460; U.S. Pat. No. 6,503,381; U.S. Pat. No. 6,461,496; U.S. Pat. No. 6,377,894; U.S. Pat. No. 6,338,790; U.S. Pat. No. 6,299,757; U.S. Pat. No. 6,284,478; U.S. Pat. No. 6,270,455; U.S. Pat. No. 6,175,752; U.S. Pat. No. 6,161,095; U.S. Pat. No. 6,144,837; U.S. Pat. No. 6,143,164; U.S. Pat. No. 6,121,009; U.S. Pat. No. 6,120,676; U.S. Pat. No. 6,071,391; U.S. Pat. No. 5,918,603; U.S. Pat. No. 5,899,855; U.S. Pat. No. 5,822,715; U.S. Pat. No. 5,820,551; U.S. Pat. No. 5,628,890; U.S. Pat. No. 5,601,435; U.S. Pat. No. 5,593,852; U.S. Pat. No. 5,509,410; U.S. Pat. No. 5,320,715; U.S. Pat. No. 5,264,014; U.S. Pat. No. 5,262,305; U.S. Pat. No. 5,262,035; U.S. Pat. No. 4,711,245; U.S. Pat. No. 4,545,382; U.S. Pat. No. 5,356,786; U.S. Pat. No. 5,543,326; U.S. Pat. No. 6,103,033; U.S. Pat. No. 6,134,461; U.S. Pat. No. 6,143,164; U.S. Pat. No. 6,144,837; U.S. Pat. No. 6,161,095; U.S. Pat. No. 6,579,690; U.S. Pat. No. 6,605,200; U.S. Pat. No. 6,605,201; U.S. Pat. No. 6,618,934; U.S. Pat. No. 6,654,625; U.S. Pat. No. 6,676,816; U.S. Pat. No. 6,730,200; U.S. Pat. No. 6,736,957; U.S. Pat. No. 6,932,892; U.S. Publication No. 2004/0186365; U.S. Publication No. 2005/0182306; U.S. Publication No. 2006/0025662; U.S. Publication No. 2006/0091006; U.S. Publication No. 2007/0056858; U.S. Publication No. 2007/0068807; U.S. Publication No. 2007/0095661; U.S. Publication No. 2007/0108048; U.S. Publication No. 2007/0199818; U.S. Publication No. 2007/0227911; U.S. Publication No. 2007/0233013; U.S. Publication No. 2008/0066305; U.S. Publication No. 2008/0081977; U.S. Publication No. 2008/0102441; U.S. Publication No. 2008/0148873; U.S. Publication No. 2008/0161666; U.S. Publication No. 2008/0267823; U.S. Publication No. 2009/0054748; U.S. patent application Ser. No. 10/745,878, filed Dec. 26, 2003 and entitled “Continuous Glucose Monitoring System and Methods of Use”, U.S. patent application Ser. No. 12/143,731, filed Jun. 20, 2008 and entitled “Health Management Devices And Methods”; U.S. patent application Ser. No. 12/143,734, filed Jun. 20, 2008 and entitled “Health Monitor”; U.S. Provisional Patent Application No. 61/149,639, filed Feb. 3, 2009 and entitled “Compact On-Body Physiological Monitoring Devices And Methods Thereof”; U.S. Provisional Application No. 61/291,326 filed Dec. 30, 2009, and U.S. Provisional Application No. 61/299,924 filed Jan. 29, 2010; U.S. patent application Ser. No. 11/461,725; U.S. patent application Ser. No. 12/131,012; U.S. patent application Ser. No. 12/242,823; U.S. patent application Ser. No. 12/363,712; U.S. patent application Ser. No. 12/698,124; U.S. patent application Ser. No. 12/698,129; U.S. patent application Ser. No. 12/714,439; U.S. patent application Ser. No. 12/794,721; U.S. patent application Ser. No. 12/842,013; U.S. patent application Ser. No. 61/238,646; U.S. patent application Ser. No. 61/345,562; U.S. patent application Ser. No. 61/361,374; and elsewhere, the disclosures of each are incorporated by reference in their entirety herein for all purposes.

The foregoing only illustrates the principles of the disclosed subject matter. Various modifications and alterations to the described embodiments will be apparent to those skilled in the art in view of the teachings herein. It will be appreciated that those skilled in the art will be able to devise numerous modifications which, although not explicitly described herein, embody the principles of the disclosed subject matter and are thus within the spirit and scope of the disclosed subject matter.

What is claimed is:

1. An inserter assembly for implanting a medical device in the skin of a subject comprising:
a housing having a proximal end and a distal end and one or more channels disposed therethrough;
a shuttle slidably received within the one or more channels of the housing, the shuttle being movable in an insertion direction;
a first driver operatively coupled to the shuttle and configured to urge the shuttle in the insertion direction;
an introducer sharp coupled to the shuttle; and
a rotatable trigger coupled to the housing and configured allow the first driver to urge the shuttle and introducer sharp in the insertion direction upon rotation of the trigger.

2. The inserter assembly of claim 1, wherein the first driver is configured with at least a compressed state and an expanded state.

3. The inserter assembly of claim 2, wherein the rotation of the trigger allows the first driver to move from the compressed state to the expanded state.

4. The inserter assembly of claim 1, wherein the housing defines a longitudinal axis extending from the distal end to the proximal end thereof, and wherein the trigger is configured for rotation about an axis substantially parallel to the longitudinal axis of the housing.

5. The inserter assembly of claim 1, wherein the housing includes a groove circumferentially disposed about the body, and the trigger includes a flange configured to engage the groove disposed about the housing.

6. The inserter assembly of claim 1, wherein the first driver comprises a spring disposed in the channel formed in the housing.

7. The inserter assembly of claim 1, wherein a second driver is disposed in the housing.

8. The inserter assembly of claim 7, wherein the second driver is configured to urge the shuttle towards a retraction position.

9. The inserter assembly of claim 1, further including a safety member to impede rotation of the trigger

10. An inserter system for implanting a medical device in the skin of a subject comprising:
an inserter assembly comprising
a housing having a proximal end and a distal end and one or more channels disposed therethrough;
a shuttle slidably received within the one or more channels of the housing, the shuttle being movable in an insertion direction;
a first driver operatively coupled to the shuttle and configured to urge the shuttle in the insertion direction;
an introducer sharp coupled to the shuttle; and
a rotatable trigger coupled to the housing and configured allow the first driver to urge the shuttle and introducer sharp in the insertion direction upon rotation of the trigger; and
a mounting unit configured to be coupled to the inserter assembly.

11. The inserter system of claim 10, wherein the distal end of the tubular housing comprises one or more latches to engage a surface of the mounting unit.

12. The inserter system of claim 10, wherein the inserter assembly is disengaged from the mounting unit by rotation of the trigger.

13. The inserter system of claim 10, wherein the mounting unit is adapted to attach to a user's body at an insertion site.

14. The inserter system of claim 10, wherein the mounting unit comprises a body, and further wherein a power supply is disposed in the body of the mounting unit.

15. The inserter system of claim 14, wherein the power supply powers an electronics unit coupled to the mounting unit.

16. The inserter system of claim 10, wherein the sharp comprises a retention structure configured to retain the medical device.

17. The inserter system of claim 10, wherein the inserter assembly further comprises a second driver is disposed in the housing.

18. The inserter system of claim 17, wherein the second driver is configured to urge the shuttle towards a retraction position.

19. The inserter system of claim 10, wherein a distal end of the housing defines an acute angle with respect to the mounting unit.

20. The inserter system of claim 10, wherein the medical device is an analyte sensor.
